(19) United States
(12) Patent Application Publication
(10) Pub. No.: US 2025/0367445 A1
(43) Pub. Date: Dec. 4, 2025

(54) CHARGING INTERFACE AND A CHARGING CONNECTOR FOR A THERAPY DEVICE

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Feng Jianxin, Shenzhen (CN); Guo Chunfang, Shenzhen (CN); BAIHUI OU, Shenzhen (CN); Lin Jiaming, Shenzhen (CN)

(73) Assignee: SHENZHEN NUON MEDICAL EQUIPMENT CO., LTD., Shenzhen (CN)

(21) Appl. No.: 19/056,808

(22) Filed: Feb. 19, 2025

(30) Foreign Application Priority Data
Jun. 3, 2024 (CN) 2024107204713
Jun. 3, 2024 (CN) 2024212531208
Jul. 23, 2024 (CN) 2024217486035
Aug. 13, 2024 (CN) 2024219569808

(51) Int. Cl. A61N 1/36; H01R 13/62; H01R 13/631

(52) U.S. Cl. CPC A61N 1/3606; H01R 13/6205; H01R 13/631; H01R 2201/12

(57) ABSTRACT

Embodiments of the present invention disclose a device for providing a therapy to a user. The device comprises a casing, at least one electro-stimulation element in the casing, a charging interface and a charging connector. The charging interface comprises: a positioning portion arranged on the casing, the positioning portion has a first positioning hole and a second positioning hole; one or more charging pins for charging the device, the one or more charging pins comprises a first electrode and a second electrode for charging, the first electrode has a first end, the first end is exposed in the first positioning hole, the second electrode has a second end, and the second end is exposed in the second positioning hole. The charging connector has a charging terminal that connects with the charging pins on the charging interface of the device.

### TECHNICAL FIELD

The present invention relates generally to a hand-held therapy device, and more particularly to a charging interface on the hand-held therapy device and a charging connector.

### BACKGROUND ART

With the improvement of people's quality of life and the change of aesthetic concepts, more and more consumers pay attention to beauty and skin care, especially women. When users carry out beauty and skin care, the combination of the liquid medicine and the electric mechanism can help the skin better absorb the nutrients in the solution, while improving the skin texture, tightening the skin, reducing pigmentation and lightening spots.

The casing of electronic therapy devices, such as phototherapy patches and instruments, is usually equipped with components such as a charging interface and switch buttons. The equipment is charged through the charging interface, and the equipment is controlled to switch on and off through the switch button. Such a structural design will cause a plurality of mounting slots to be set up on the casing to correspond to the installation of the charging interface and switch buttons, resulting in complex overall structure, low assembly efficiency, and affecting the overall aesthetics.

Furthermore, a charging connector is used to charge the electronic equipment. The charging connector usually includes male and female electrical connectors, and the corresponding device's signal transmission or electrical connection is realized through the contact between the male and female terminals.

However, after the terminals of the male connector and the terminal of the female electrical connector are docked, there is a hidden danger of poor connection stability, especially in the use scenario where the electrical connector is thin and short.

### OBJECTS OF THE INVENTION

Some of the objects of the invention are as follows:

An object of the present invention is to provide a therapy device having a charging interface arranged on a switch device.

Another object of the present invention is to provide a therapy device with the same mounting slot for mounting a charging interface and a switch to provide a therapy device with a simple, aesthetically pleasing structure.

Another object of the present invention is to provide a charging interface and a charging connector for a therapy device with good connection stability between them.

Another object of the present invention is to provide a charging connector for a therapy device which can dock with different types of shapes of the therapy device, thereby improving the versatility of the charging connector.

### SUMMARY OF THE INVENTION

According to a first aspect of the present invention, a system for providing a therapy to a user is provided. The system comprising: a therapy device having one or more electro-stimulation element; a charging interface in the therapy device, the charging interface comprising: a positioning portion arranged on surface of the therapy device, the positioning portion has one or more positioning hole for positioning a first electrode and a second electrode; a charging connector having a charging terminal that connects with the first electrode and the second electrode on the charging interface of the therapy device for charging the therapy device; and wherein the positioning portion act as an activating switch for controlling the one or more electro-stimulation element.

In one embodiment of the invention, the electro-stimulation element includes but is not limited to a stimulation element such as Light Emitting Diodes (LEDs), lasers, heating elements, cooling elements, vibration elements, electrodes, micro-current element, an actuator, a pump, a motor, a vibrator, a heater, a piezoelectric element, an abrasive element.

In one embodiment of the invention, the charging interface comprises a magnetic suction piece and the charging connector comprises a magnetic suction structure to magnetically attach to the charging interface when docked.

According to a second aspect of the present invention, a device for providing a therapy to a user is provided. The device comprising: one or more electro-stimulation element; a circuit board electrically connected with the one or more electro-stimulation element; a charging interface, the charging interface comprising: a positioning portion having one or more positioning hole; an accommodating cavity; one or more charging pins in the accommodating cavity for charging the device, the one or more charging pins comprises a first electrode and a second electrode for charging; and wherein the positioning portion is designed to form a conductive loop between the circuit board and the one or more electro-stimulation element, thereby activating the one or more electro-stimulation element.

In one embodiment of the invention, the circuit board is electrically connected with the first electrode and the second electrode, and is used to form a conductive loop when the first electrode and the second electrode are in contact with skin.

In one embodiment of the invention, the positioning portion is configured to switch between a first position where the first electrode and the second electrode are in the accommodating cavity, and a second position where the first electrode and the second electrode are exposed to the one or more positioning hole.

In one embodiment of the invention, the positioning portion is provided in a casing having a slot and the positioning portion comprise a first positioning hole and a second positioning hole, and the positioning portion comprises: a positioning wall in the first positioning hole and the second position hole; a periphery wall configured to move telescopically in the slot; the periphery wall is connected to an edge of the positioning wall to construct the accommodating cavity, and the accommodating cavity is provided with an opening for the circuit board; and wherein the positioning wall is configured to move fro and towards the circuit board.

In one embodiment of the present invention, an elastic part is provided between the positioning wall and the circuit board.

In one embodiment of the present invention, an inner edge of the opening is provided with a first stop structure on one side of the circuit board, and an outer edge of the periphery wall is provided with a second stop structure.

In one embodiment of the invention, the device further comprising a switch piece connected to the circuit board.

In one embodiment of the invention, the positioning portion comprises a boss, wherein when the positioning portion is pressed, the boss presses the switch piece to form a conductive loop between the circuit board and the one or more electro-stimulation element.

In one embodiment of the invention, the charging interface further comprises an elastic reset piece connected with a mounting pad, the mounting pad has a movable port for accommodating the positioning portion.

In one embodiment of the invention, the positioning portion has a limiting groove and the mounting pad has a limiting portion for inserted in the limiting groove.

In one embodiment of the invention, the positioning portion has one or more magnetic suction piece.

In one embodiment of the invention, the charging pins are configured to connect to a charging connector for charging the device.

According to a third aspect of the present invention, a charging connector for charging a device for providing therapy to a user is provided. The charging connector comprising: a shell; an electrical connection assembly comprises a charging terminal, the charging terminal has a first end in the shell and a second end at a face of the shell; a magnetic suction structure in the shell or at the charging terminal; wherein the shell comprises a storage chamber and a recessed portion having two mounting holes, the recessed portion comprises a first groove and a second groove; and wherein the charging terminal is configured to connect to a charging pin on a charging interface of the device.

In one embodiment of the invention, the shell has a first end plate for butting with a charging interface on the device and the magnetic suction structure is positioned at the first end plate.

In one embodiment of the invention, the shell has a second end plate opposite to the first end plate, the second end plate has an indicator light connected with the charging terminal.

In one embodiment of the invention, the first groove is a circular groove, the second groove is connected to periphery of the first groove, and the mounting hole penetrates through the first groove and communicates with the storage chamber.

In one embodiment of the invention, the second grooves are two in number, and the center of both the second grooves is collinear with the center of the first groove.

In one embodiment of the invention, contour of the second groove is arc-shaped.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-a (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other component on the piece.

In the context of the specification, the terms “first”, “second” and “third” are only used for descriptive purpose and does not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and

Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a charging interface on a hand-held therapy device, in accordance with an embodiment of the present invention.

FIG. 2 and FIG. 3 shows a cross-section view of a positioning portion of the charging interface, in accordance with an embodiment of the present invention.

FIG. 4 shows the charging interface docked with a charging connector, in accordance with an embodiment of the present invention.

FIG. 5 shows a cross-sectional view of the charging interface, in accordance with an embodiment of the present invention.

FIG. 6 shows an exploded view of the charging interface, in accordance with an embodiment of the present invention.

FIG. 7 shows a cross-sectional view of a therapy device having the charging interface along the horizontal axis, in accordance with an embodiment of the present invention.

FIG. 8 shows a cross-sectional view of a therapy device having the charging interface along the horizontal axis, in accordance with an embodiment of the present invention.

FIG. 9 illustrates a charging connector, in accordance with an embodiment of the present invention.

FIG. 10 shows an exploded view of the charging connector, in accordance with an embodiment of the present invention.

FIG. 11 shows a cross-sectional view of the charging connector with magnetic suction piece installed inside the shell, in accordance with an embodiment of the present invention.

FIG. 12 shows a cross-sectional view of the charging connector with magnetic suction piece installed on top of the shell, in accordance with an embodiment of the present invention.

FIG. 13 shows a cross-sectional view of the charging connector with magnetic suction piece installed between the shell layer, in accordance with an embodiment of the present invention.

FIG. 14 shows a cross-sectional view of the charging connector with magnetic suction piece installed along the charging terminal, in accordance with an embodiment of the present invention.

FIG. 15 shows a cross-sectional view of the charging connector with magnetic suction piece installed inside the charging terminal, in accordance with an embodiment of the present invention.

FIG. 16 shows a cross-sectional view of the charging connector with magnetic suction piece installed at top of the charging terminal, in accordance with an embodiment of the present invention.

FIG. 17 shows the charging connector in form of a charging dock, in accordance with an embodiment of the present invention.

FIG. 18 chows a charging connector that can be docked with a universal-shaped therapy device, in accordance with an embodiment of the present invention.

FIG. 19 to FIG. 21 are the planar diagram of the charging connector showing different type of shapes that can be docked with the charging connector, in accordance with an embodiment of the present invention.

FIG. 22 and FIG. 23 shows cross-sectional views of the charging connector along vertical axis, in accordance with an embodiment of the present invention.

FIG. 24 shows a perspective view of the charging connector, in accordance with an embodiment of the present invention.

FIG. 25 shows a front planar view of the charging connector, in accordance with an embodiment of the present invention.

FIG. 26 shows a side cross-sectional view of the charging connector along vertical axis, in accordance with an embodiment of the present invention.

FIG. 27 shows a front cross-sectional view of the charging connector along vertical axis, in accordance with an embodiment of the present invention.

FIG. 28 shows a planar view of the charging connector in FIG. 27, in accordance with an embodiment of the present invention.

FIG. 29 shows a cross-sectional view of a charging connector having a magnetic suction piece, in accordance with an embodiment of the present invention.

FIG. 30 shows a planar view of the charging connector of FIG. 29, in accordance with an embodiment of the present invention.

FIG. 31 shows a cross-sectional view of the charging connector with magnetic suction piece installed in the shell along a charging pin, in accordance with an embodiment of the present invention.

FIG. 32 and FIG. 33 shows a therapy device with a charging interface that can be connected with the charging connector, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a bottle cover. The bottom cover comprises a housing, a mounting seat and an elastic member. The housing has a first end for detachable connection with the container body and a second end opposite the first. The housing has an accommodating chamber that runs through the first end and the second end. The mounting seat is mounted in the accommodating chamber and the elastic member is used to push the mounting seat towards the second end. The housing is provided with a first locking bit for locking the mounting seat when the mounting seat moves to the first end, a second locking bit for locking the mounting seat when the mounting seat moves to the second end, a first guiding structure for guiding the mounting seat to lift and a first guiding structure for guiding the mounting seat to rotate between the first locking bit and the second locking bit. The bottle cover provided in the present application can solve the technical problem that the mounting seat reset is inconvenient that exists in the related art.

In an embodiment of the present invention, a charging interface on a hand-held therapy device that provides alternate therapy to a user is provided. The charging interface comprises a positioning portion arranged on casing of the therapy device. The positioning portion is provided with an accommodating cavity, and a first positioning hole and a second positioning hole are connected to the accommodating cavity. The charging pins are arranged in the accommodating cavity. The charging pins comprises a first electrode and a second electrode for charging. The first electrode has a first end exposed in the first positioning hole. The second electrode has a second end exposed in the second positioning hole. The therapy device has a circuit board that is electrically connected to the one or more electro-stimulation elements, the first electrode, and the second electrode. A conductive circuit is formed when the first end of the first electrode and the second end of the second electrode come into contact with the skin. The application can realize multiple functions such as charging, switching on and off the therapy device, simplifying the structure of the whole machine, and improving its aesthetics.

Several embodiments of the present invention will now be described in detail with references to FIGS.

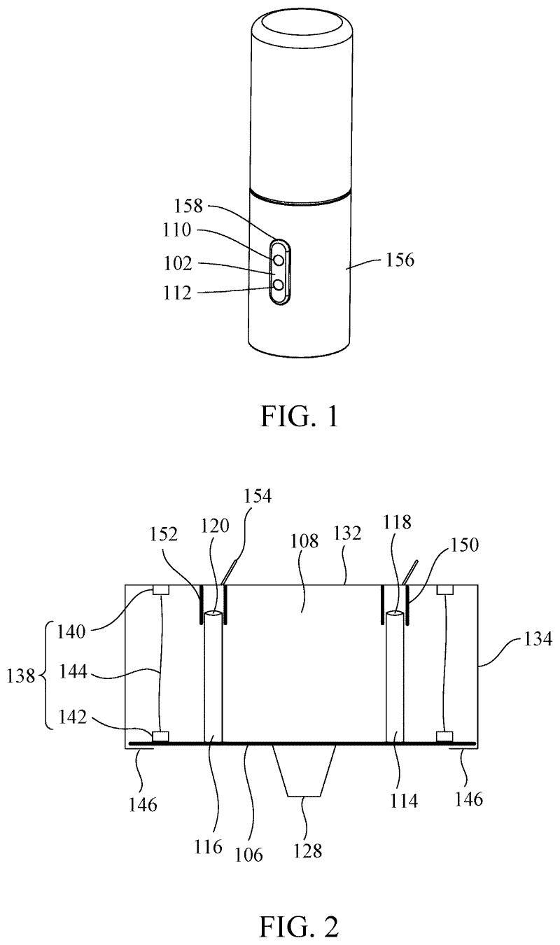

Referring to FIG. 1 to FIG. 3, a charging interface provided on a casing of a hand-held therapy device is provided. The charging interface 100 comprises a positioning portion 102 provided on the casing 156 of the therapy device, charging pins 104 and a circuit board 106. The hand-held therapy device can be used for providing alternate therapy to the user. The therapy device may comprise one or more electro-stimulation element connected to a battery. The one or more electro-stimulation element may include but is not limited to a stimulation element such as Light Emitting Diodes (LEDs), lasers, heating elements, cooling elements, vibration elements, electrodes, micro-current element, an actuator, a pump, a motor, a vibrator, a heater, a piezoelectric element, an abrasive element. The positioning portion 102 is arranged on the casing 156, and the positioning portion 102 is provided with an accommodating cavity 108. A first positioning hole 110 and a second positioning hole 112 are connected with the accommodating cavity 108 and the charging pins 104 are arranged in the accommodating cavity 108. The charging pins 104 comprises a first electrode 114 and a second electrode 116 for charging. One of the first electrodes 114 and the second electrode 116 is a positive electrode, and the other is a negative electrode. The first electrode 114 has a first end 118, and the first end 118 is exposed (convex or flush) in the first positioning hole 110. The second electrode 116 has a second end 120, and the second end 120 is exposed (protruding or flush) in the second positioning hole 112. The circuit board 106 is electrically connected with the first electrode 114 and the second electrode 116. The circuit board 106 is used to form a conductive loop when the first end 118 of the first electrode 114 and the second end 120 of the second electrode 116 are in contact with the skin.

In an embodiment of the present invention, the charging pins may be arranged in the positioning hole to form either a male or female socket. Accordingly, the charging connector has male-type or female-type charging terminals corresponding to the type on the charging interface.

In this embodiment of the present application, the aim is to simplify the design of the phototherapy device and improve the user's experience. Specifically:

The casing 156 is mainly used to carry each component as the shell of the whole therapy device, and the electro-stimulation element is electrically connected with the circuit board 106. The electro-stimulation element can be a light-emitting diode that can emit light outward through a translucent portion, thereby allowing phototherapy to be done.

The positioning portion 102 is provided with the accommodating cavity 108 for accommodating the charging pins 104. The charging pins 104 comprises a first electrode 114 and a second electrode 116, and the two electrodes are exposed in the first positioning hole 110 and the second positioning hole 112 of the positioning portion 102 respectively through their respective ends (first end 118 and second end 120). This design allows two electrodes to be in contact with positive charging contact 124 and negative charging contact 126 on the charging connector 122 for charging, and also allows the two electrodes to be in direct contact with the user's skin.

In an embodiment, the circuit board 106 can be arranged in the accommodating cavity 108 of the positioning portion 102. A fixing frame 128 can be arranged at the bottom of the circuit board 106, and the circuit board 106 is fixed in the casing 156 through the fixing frame 128. The first electrode 114 and the second electrode 116 can be fixed on the circuit board 106, or, the circuit board 106 is fixed in the casing 156 through the fixing frame 128, and the first electrode 114 and the second electrode 116 can be fixed on structures such as the casing 156 or the brackets in the casing 156. The circuit board 106 is electrically connected with the first electrode 114 and the second electrode 116 to detect the contact state between the two electrodes and the skin and trigger the start-up or shutdown action of the phototherapy device accordingly. When the two electrodes come into contact with the skin, a closed loop is formed, and the circuit board 106 is able to detect this change and trigger the phototherapy device to turn on.

Referring to FIG. 4, when the therapy device is placed on the charging connector 122, the first electrode 114 and the second electrode 116 are in contact with the positive charging contact 124 and the negative charging contact 126 on the charging connector 122 through the first positioning hole 110 and the second positioning hole 112, and the charging contact provides power to the electrode to realize the charging of the phototherapy device.

When the user contacts the therapy device to the skin, the first electrode 114 and the second electrode 116 are in contact with the skin, and because the skin is a conductive body, a closed conductive circuit is formed between the two electrodes, the circuit board 106 and the skin. After the circuit board 106 detects this conductive loop signal, it triggers the therapy device to turn on and generates an appropriate microcurrent between the two electrodes to stimulate the skin to achieve therapy by the electro-stimulation element.

When the user removes the therapy device from the skin, the loop is disconnected, and the circuit board 106 detects this change and triggers the device to shut down. In this way, the user can control the power on and off of the therapy device through simple skin contact, which is more intuitive and convenient to operate.

The hand-held therapy device provided in the embodiment of the present application, on the one hand, can realize the therapy function, and on the other hand, through the charging pins 104 integrated charging and on/off dual functions in one. The hand-held therapy device does not need additional switching buttons and mounting grooves, thereby making the therapy device more concise and beautiful, and improving the user's operation convenience and overall experience.

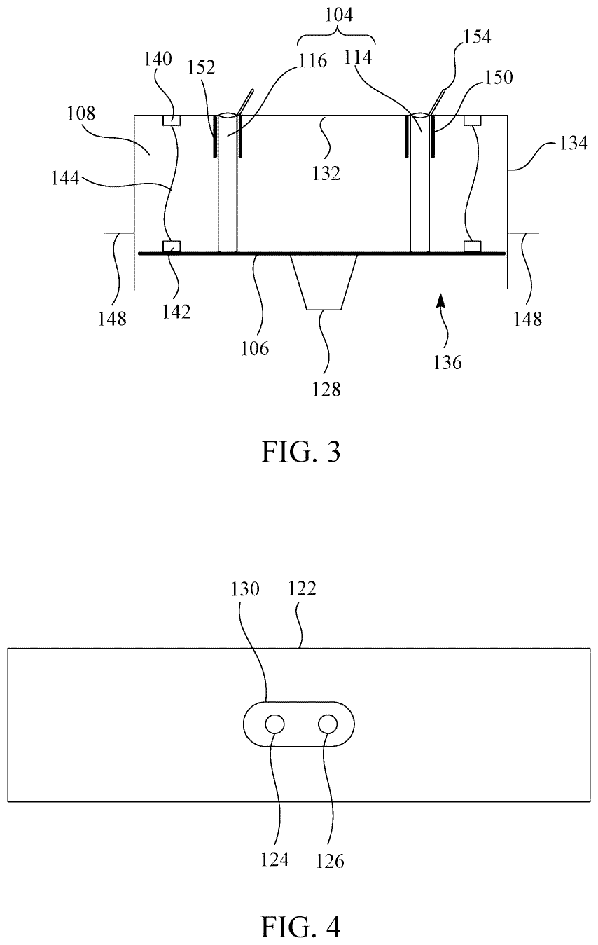

Referring to FIG. 2 and FIG. 3, the positioning portion 102 can move telescopically relative to the first electrode 114 and the second electrode 116 to switch between the first position and the second position. When the positioning portion 102 moves to the first position, the first end 118 of the first electrode 114 and the second end 120 of the second electrode 116 are all built into the accommodating cavity 108. When the positioning portion 102 moves to the second position, the first end 118 of the first electrode 114 and the second end 120 of the second electrode 116 are respectively exposed to the first positioning hole 110 and the second positioning hole 112.

In an embodiment of the present application, the positioning portion 102 is designed to be retractable and mobile, capable of switching between the first position and the second position. When in the first position, the first electrode 114 and the second electrode 116 are completely hidden in the accommodating cavity 108 of the positioning portion 102 and are not exposed. When in the second position, the first end 118 of the first electrode 114 and the second end 120 of the second electrode 116 are respectively exposed in the first positioning hole 110 and the second positioning hole 112 of the positioning portion 102, so that it can contact with the skin or be charged.

Switch-on process: when the user adheres the therapy device to the skin, the positioning portion 102 is squeezed by the skin and moves to the second position so that the first electrode 114 and the second electrode 116 stretch out relative to each other and touch the skin. A closed conductive loop is formed between the two electrodes, the circuit board 106 and the skin, and the circuit board 106 detects this conductive circuit signal, triggers the device to turn on, and generates an appropriate microcurrent between the two electrodes to stimulate the skin for physical therapy.

When the user moves the therapy device away from the skin, the positioning portion 102 automatically resets to the first position because of the loss of squeezing force, and the two electrodes are then retracted relative to each other into the accommodating cavity 108 of the positioning portion 102, and the circuit board 106 detects that the circuit is disconnected, triggers the equipment to shut down. Therefore, when the therapy device is not in use, the charging pins 104 can be completely hidden in the inside of the positioning portion 102, which can avoid the user from accidentally touching the charging pins 104 and cause start-up, and can effectively protect the charging pins 104 and prevent it from being damaged, thereby prolonging the service life.

Charging process: Referring to FIG. 4, the top of the charging connector 122 is provided with a groove 130 that is matched with the positioning portion 102, and the positive charging contact 124 and the negative charging contact 126 of the charging connector 122 are located at the bottom of the groove 130. When the therapy device is placed on the charging connector 122, the positioning portion 102 is positioned in the groove 130 but does not contact the bottom of the groove 130, so it will not be extruded and moved. The positioning portion 102 and the charging pins 104 do not change relative position at this moment, the positioning portion 102 and the groove 130 cooperate to realize positioning, and the positive charging contact 124 and the negative charging contact 126 of the charging connector 122 are respectively in contact with the corresponding first electrode 114 and the second electrode 116 through the first positioning hole 110 and the second positioning hole 112 of the positioning portion 102 to realize charging. This design can effectively improve the user's experience.

In some embodiments, the top of the charging connector 122 is a planar structure. When the therapy device is placed on the charging connector 122, the positioning portion 102 is extruded and moved by being extruded and moved by contact with the top plane of the charging connector 122, and the first electrode 114 and the second electrode 116 are relatively stretched out and exposed in the first positioning hole 110. At this moment, the second positioning hole 112 of the positioning portion 102, are in contact with the positive charging contact 124 and the negative charging contact 126 of the charging connector 122 to realize charging. This design can ensure that the charging pins 104 are in better contact with the charging contact, improving the charging reliability.

Referring to FIG. 1, FIG. 2 and FIG. 3, the casing 156 is provided with a slot 158. The positioning portion 102 comprises a positioning wall 132 and a periphery wall 134. The positioning wall 132 is provided with a first positioning hole 110 and a second positioning hole 112. The periphery wall 134 can expand and contract in the slot 158. The periphery wall 134 is vertically connected with the edge of the positioning wall 132 to construct the accommodating cavity 108, and an opening 136 is provided on one side of the accommodating cavity 108 relative to the positioning wall 132. The circuit board 106 is arranged at the opening 136, and the positioning wall 132 can expand and contract in the direction of being far away from or close to the circuit board 106. The circuit board 106 is provided with the first electrode 114 and the second electrode 116.

Specifically, when the user adheres the therapy device to the skin, the positioning wall 132 is subjected to a squeezing force and moves close to the circuit board 106 to the second position, so that the first electrode 114 and the second electrode 116 extend out relative to each other and touch the skin.

A closed conductive loop is formed between the two electrodes, the circuit board 106 and the skin, and the circuit board 106 detects this change and triggers the device to turn on and generates an appropriate micro-current between the two electrodes to start stimulating the skin by the electro-stimulation element.

When the user moves the device away from the skin, the positioning wall 132 is far away from the circuit board 106 because of the loss of extrusion force, automatically resets to the first position, and the charging pins 104 is then relatively retracted into the accommodating cavity 108, and the circuit board 106 detects that the circuit is disconnected, and triggers the electro-stimulation element to shut down.

The circuit board 106 is arranged at the opening 136 of the positioning portion 102, which effectively utilizes the space, and can make the overall structure of the device more compact.

Referring to FIG. 2 and FIG. 3, an elastic structure 138 is arranged between the positioning wall 132 and the circuit board 106. The elastic structure 138 can be a spring or a shrapnel.

In an embodiment, the positioning wall 132 can be telescopic and move relative to the circuit board 106. The elastic structure 138 is located between the positioning wall 132 and the circuit board 106. The effect of the elastic structure 138 is that when the positioning wall 132 is detached from the skin or the charging connector 122, it automatically helps the positioning portion 102 reset to the initial position (first position). The charging pins 104 are hidden in the positioning portion 102. This design ensures that the charging pins 104 are protected when not in use, while also keeping the device neat and aesthetically pleasing.

Specifically, when the positioning wall 132 is subjected to an extrusion force (such as contact with the skin), the positioning wall 132 will move in a direction close to the circuit board 106, to the second position, so that the first electrode 114 and the second electrode 116 extend out relative to each other and touch the skin.

When the positioning wall 132 is detached from the skin, the recovery force provided by the elastic structure 138 causes the positioning wall 132 to move in a direction away from the circuit board 106, to the first position, and the charging pins 104 then retract into the accommodating cavity 108.

This realizes the automatic reset function of the positioning portion 102 by arranging an elastic structure 138 between the positioning wall 132 and the circuit board 106, and ensures that the charging pins 104 can be safely hidden when not in use. This design not only simplifies the user's operation, but also enhances the durability and aesthetics of the product.

Referring to FIG. 2 and FIG. 3, the elastic structure 138 comprises: a first connecting portion 140, a second connecting portion 142 and an elastic part 144. The first connecting portion 140 is located on the positioning wall 132 and the second connecting portion 142 is arranged on the circuit board 106. The two ends of the elastic part 144 are connected with the first connecting portion 140 and the second connecting portion 142.

In some instances, the first connecting portion 140 and the second connecting portion 142 may be designed as connecting posts. The elastic part 144 is a spring, and the two ends of the spring are respectively sleeved on two connecting posts.

In some instances, the first connecting portion 140 and the second connecting portion 142 can be designed as a clamping part, an adhesive part, a weldment part, or the like. The elastic part 144 is a shrapnel, and the two ends of the shrapnel are respectively connected with the first connecting portion 140 and the second connecting portion 142 by means of snapping, gluing or welding.

Referring to FIG. 2 and FIG. 3, the elastic structure 138 ensures the reliability of elastic resent. The number of elastic structures 138 is at least two groups, and each group of elastic structures 138 is arranged at intervals. For instance, the two groups of elastic structures 138 are arranged on the left and right sides of the charging pins 104.

When the number of elastic structures 138 is at least three groups, each group of elastic structures 138 is uniformly arranged in a circular direction around the charging pins 104.

Such design can effectively improve the moving stability of the positioning portion 102, thereby ensuring that the charging pins 104 can accurately extend into the positioning hole structure and ensure the reliability of charging and switching on/off.

Referring to FIG. 2, the inner edge of the opening 136 of the positioning portion 102 is provided with a first stop structure 146, and the first stop structure 146 is arranged on one side of the circuit board 106 back to the positioning wall 132. The first stop structure 146 can be in plurality of number. When the first stop structure 146 comprises at least two first stop blocks of the split structure, each first stop block is arranged at intervals along the circumferential direction of the inner edge of the opening 136 of the positioning portion 102.

The first stop structure 146 is the first stop ring of the integrated structure, and the first stop ring is located at the inner edge of the opening 136.

When the positioning portion 102 moves to the initial position (first position), the first end 118 of the first electrode 114 and the second end 120 of the second electrode 116 are all built in the accommodating cavity 108, and the first stop structure 146 can hook the circuit board 106 at this moment, and the positioning portion 102 is prevented from falling off from the circuit board 106, thereby improving the stability of the therapy device.

Referring to FIG. 3, the outer side of the periphery wall 134 of the positioning portion 102 is provided with a second stop structure 148, and the second stop structure 148 is located in the slot 158 of the casing 156.

As shown in FIG. 1, the casing 156 of the phototherapy device is provided with a slot 158 matched with the positioning portion 102, and the positioning portion 102 is arranged in the slot 158.

The second stop structure 148 comprises at least two second stop blocks of the split structure, and each second stop block is arranged at intervals along the circumferential direction of the edge of the slot 158 and is clamped in the slot 158.

In some instance, the second stop structure 148 is the second stop ring of the integrated structure, and the second stop ring is clamped in the slot 158.

When the positioning portion 102 moves to the initial position (first position), the first end 118 of the first electrode 114 and the second end 120 of the second electrode 116 are all built into the accommodating cavity 108, and the second stop structure 148 is stucked to the edge of the slot 158 at this moment, and the positioning portion 102 is prevented from falling off from the casing 156, thereby improving the stability of the equipment.

Referring to FIG. 2 and FIG. 3, the inner side of the positioning wall 132 is provided with a first guide sleeve 150 and a second guide sleeve 152. The first guide sleeve 150 is in communication with the first positioning hole 110 and is used for guiding the first end 118 of the first electrode 114. The casing 156 is in communication with the second positioning hole 112 and is used to guide the second end 120 of the second electrode 116.

Specifically, the first end 118 of the first electrode 114 and the second end 120 of the second electrode 116 pass through the first guide sleeve 150 and the second guide sleeve 152, respectively and carry out telescopic movement through the first guide sleeve 150 and the second guide sleeve 152 relative positioning portion 102.

When the positioning portion 102 moves to the first position, the first end 118 of the first electrode 114 and the second end 120 of the second electrode 116 are built into the corresponding guide sleeve and are not exposed.

When the positioning portion 102 moves to the second position, the first end 118 of the first electrode 114 and the second end 120 of the second electrode 116 stretch out from the corresponding guide sleeve respectively so that it can be in contact with the skin or be charged.

This ensures the stability of the charging pins 104 when it expands and contracts in the positioning hole structure through the guide sleeve structure, reduces the shaking, and ensures the stability and reliability of the equipment in charging and switching on and off. The guide sleeve structure makes the charging pins 104 expand and contract quickly and smoothly, and ensures that the therapy device can respond quickly to the user's operation. In addition, the movement of the charging pins 104 is guided by the guide sleeve structure, the friction between the charging pins 104 and the positioning wall 132 can be reduced, the wear is reduced, and the device's service life is prolonged.

Referring to FIG. 2 and FIG. 3, the first positioning hole 110 and the second positioning hole 112 are provided with caps 154, and the caps 154 are detachably connected or movably connected with the positioning portion 102.

The cap 154 may be a detachable split structure with the positioning portion 102, and may also be movably connected to the edge of the corresponding positioning hole through a flexible belt or a pin shaft. When the equipment needs to be used, the cap 154 can be removed or turned over, and when not in use, the cap 154 can be covered to protect the charging pin.

Specifically, the cap 154 is independent of the positioning portion 102, and when the equipment needs to be used, the cap 154 can be removed manually. When not in use, caps 154 protect the charging pins 104.

The cap 154 is movably connected with the edge of the positioning hole by a flexible belt (such as a plastic belt, a rubber belt) or a pin shaft. When the equipment needs to be used, the cap 154 is turned over so that it does not affect the protrusion of the charging pins 104. When not in use, cover the cap 154 directly to protect the charging pins 104. At the same time, the movable connection design can prevent the loss of cap 154, improving the device's overall durability.

Therefore, the embodiment of the present application can effectively protect the charging pins 104 from the pollution of dust and impurities through the cap 154, and prolong the service life of the equipment.

Referring to FIG. 1, the casing 156 is the main shell of the therapy device. The casing 156 is provided with a slot 158 matched with the positioning portion 102. The slot 158 on the casing 156 is used for accommodating the positioning portion 102, and when the positioning portion 102 is subjected to a squeezing force, it can be retracted into the slot 158. The design of the slot 158 is such that the positioning portion 102 can be moved between the first position and the second position to realize the expansion and contraction of the charging pins 104.

When the positioning portion 102 is in the initial position (first position), the positioning portion 102 protrudes out of the slot 158, and the first end 118 of the first electrode 114 and the second end 120 of the second electrode 116 are built into the positioning portion 102 at this moment.

When the positioning portion 102 is subjected to extrusion force, the positioning portion 102 retracts back into the slot 158, that is, moves to the second position, and the first end 118 of the first electrode 114 and the second end 120 of the second electrode 116 stretch out from the first positioning hole 110 and the second positioning hole 112 of the positioning portion 102 respectively, so as to facilitate charging or contact with the skin, and realize the switch.

In another embodiment of the present invention, a charging interface in a therapy device is provided. The charging interface comprises a positioning portion and two charging pins. The positioning portion has a first surface that can be pressed by a user, and a second surface that is opposite to the first surface. The positioning portion is provided with a mounting hole, and the mounting hole runs through the first surface and the second surface. The charging pins penetrate through the mounting hole, one end of the charging pins is exposed on the first surface, and the other end of the charging structure is exposed on the second surface. The charging pins are integrated on the positioning portion, therefore, the hole position for assembling the positioning portion needs to be reserved on the housing of the therapy device.

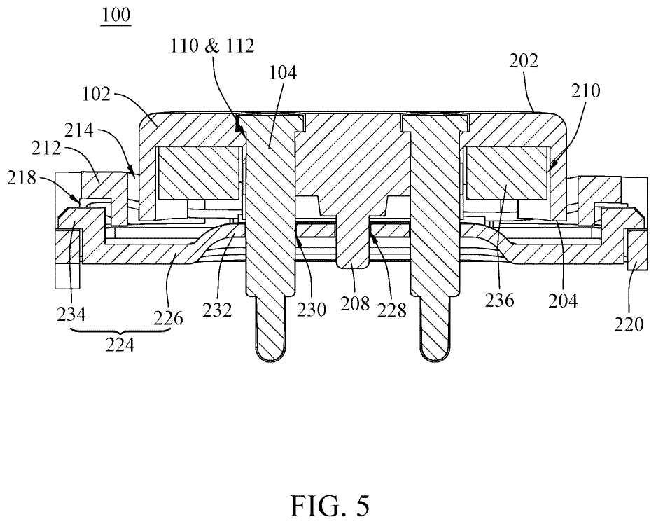

Referring to FIG. 1, FIG. 5 to FIG. 8, the charging interface 100 is mainly applied to the hand-held electrical device, and specifically, the charging interface 100 is used for assembling on the casing 156 of the hand-held electric device.

Referring to FIG. 5 to FIG. 7, the charging interface 100 comprises a positioning portion 102 and charging pins 104, and the positioning portion 102 is used to trigger the switch piece 250 on an electrical mechanism. The positioning portion 102 has a first surface 202 for pressing and a second surface 204 that is opposite the first surface 202. The first surface 202 and the second surface 204 are arranged against each other, and the positioning portion 102 is provided with a first positioning hole 110 and a second positioning hole 112. The first positioning hole 110 and the second positioning hole 112 penetrate through the first surface 202 and the second surface 204. The charging pins 104 penetrates through the first positioning hole 110 and the second positioning hole 112. One end of the charging pins 104 is exposed on the first surface 202, and the other end of the charging pins 104 is exposed on the second surface 204.

When the positioning portion 102 of the charging interface 100 of the application is installed in the casing 156 of the therapy device, the first surface 202 is oriented to the outside of the casing 156 for the user to press, and the second surface 204 is oriented towards the casing 156. The positioning portion 102 is correspondingly arranged with the switch piece 250 on the electrical mechanism of the therapy device. The positioning portion 102 is triggered by pressing the first surface 202 of the positioning portion 102, and one end of the charging pins 104 is exposed to the first surface 202 through pressing. The charging cable can be electrically connected with the charging pins 104, and the other end of the charging pins 104 is exposed to the second surface 204, so that the charging pins 104 can be electrically connected with the circuit board 106 in the casing 156.

The charging pins 104 and the positioning portion 102 combinedly form the charging interface 100 and only the hole position for assembling the positioning portion 102 needs to be provided on the casing 156. The charging pins 104 is penetrated in the first positioning hole 110 and the second positioning hole 112, and the charging pins 104 is integrated on the positioning portion 102. The hole position of assembling the charging pins 104 is not reserved, as long as the positioning portion 102 is assembled on the casing 156. By integrating the charging pins 104 on the positioning portion 102, the charging pins 104 does not need to occupy the extra area of the outer surface of the casing 156, effectively reduces the occupied area of the outer surface of the casing 156, makes the appearance of the casing 156 more tidy, and the aesthetics are better.

In an optional embodiment, the charging pins 104 is sealed and threaded into the first positioning hole 110 and the second positioning hole 112. For instance, the charging pins 104 is penetrated into the first positioning hole 110 and the second positioning hole 112. Then the sealing block, sealing strip, etc. are filled between the charging pins 104 and the first positioning hole 110 and the second positioning hole 112, or the shell of the charging pins 104. The positioning portion 102 are integrally injection molded.

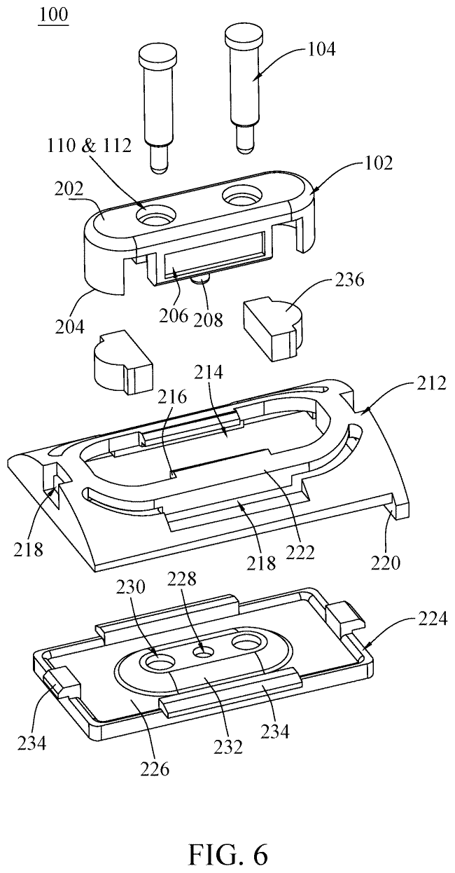

Referring to FIG. 5 and FIG. 6, the charging interface 100 further comprises a mounting pad 212 and an elastic reset piece 224. The mounting pad 212 is provided with a movable port 214, the positioning portion 102 is movably worn in the movable port 214, and the elastic reset piece 224 is connected with the mounting pad 212.

The mounting pad 212 is used for being installed on the casing 156 of the therapy device, and the positioning portion 102 is assembled on the casing 156 of the electrical mechanism through the mounting pad 212.

Through the positioning portion 102 movable wear is arranged in the movable port 214. When the positioning portion 102 is pressed, the positioning portion 102 is close to the switch piece 250, and the switch piece 250 is triggered. The positioning portion 102 is promoted to produce elastic deformation simultaneously. After the pressing force of the positioning portion 102 is revoked, the positioning portion 102 is far away from the switch piece 250 under the effect of the elastic reset piece 224, and the positioning portion is reset. The overall structure is simple, and the assembly is convenient.

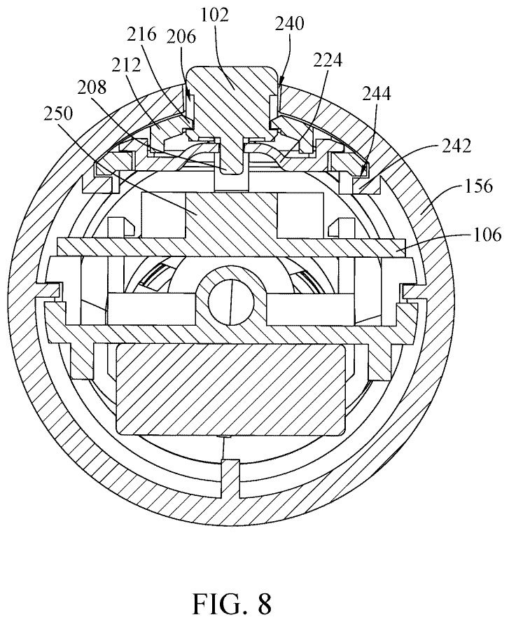

Referring to FIG. 6 and FIG. 8, a limiting groove 206 is formed in the positioning portion 102, and a limiting portion 216 is arranged on the mounting pad 212 to insert into the limiting groove 206. The limiting portion 216 is limited to be located between the opposite two sides of the wall of the limiting groove 206 along the thickness direction of the positioning portion 102.

When the positioning portion 102 is pressed to make the positioning portion 102 move in the direction of the switch piece 250, the limiting portion 216 relatively moves in the limiting groove 206 along the thickness direction of the positioning portion 102. When the positioning portion 102 triggers the switch piece 250, the limiting portion 216 is butted against the side wall of the limiting groove 206 close to the first surface 202, the positioning portion 102 movement is limited, and the positioning portion 102 is avoided from crushing the switch piece 250. In the process of reviving the pressing force of positioning portion 102, so that positioning portion 102 is far away from the switch piece 250 under the elastic force of elastic reset piece 224, the limiting portion 216 relatively moves in the limiting groove 206 along the thickness direction of positioning portion 102, until the limiting portion 216 is butted against the side of the limiting groove 206 close to the second surface 204. The positioning portion 102 movement is limited, the positioning portion 102 is avoided from being separated from the mounting pad 212, and the positioning portion 102 is avoided from shaking relative to the mounting pad 212 simultaneously.

During the movement of the positioning portion 102, the cooperation between the limiting portion 216 and limiting groove 206 ensures that the motion range of positioning portion 102 is limited, and the positioning portion 102 is prevented from crushing the switch piece 250 or detached from the mounting pad 212, and the positioning portion 102 is avoided from shaking relative to the mounting pad 212 simultaneously.

The positioning portion 102 may have two or more limiting groove 206. For example, the number of limiting groove 206 is two, and the two limiting groove 206 are respectively arranged on two opposite sides of positioning portion 102. Similarly, the mounting pad may have two or more limiting portion 216. For example, the number of limiting portion 216 is two, and the two limiting portion 216 are respectively positioned at the opposite two sides of movable port 214. The two limiting portion 216 are inserted into two limiting groove 206 respectively. The presence of two limiting portion 216 with two limiting groove 206, is conducive to improving the stability of limiting the movement of the positioning portion 102, and effectively prevents the positioning portion 102 from shaking or deflecting in the movement process.

Referring to FIG. 5 and FIG. 6, the elastic reset piece 224 comprises a sealing plate 226 and a convex 234. The sealing plate 226 is used for matching the movable port 214. The convex 234 is located at the edge of the sealing plate 226, and a clamping bit 218 is provided in the mounting pad 212. The convex 234 is clamped into the clamping bit 218 in conjunction with the clamping position. The edge of the sealing plate 226 is connected with the mounting pad 212 to form an integral structure. The elastic reset piece 224 can be a silicone part or the like.

The sealing plate 226 can be located on the inner side of the movable port 214. The edge of the sealing plate 226 is connected with the mounting pad 212, so that the sealing plate 226 seals the movable port 214. The sealing plate 226 is against the positioning portion 102, and the sealing plate 226 can produce elastic deformation under stress, so that when the positioning portion 102 is pressed, the positioning portion 102 can promote the sealing plate 226 to produce elastic deformation. After the pressing force on the positioning portion 102 is revoked, the sealing plate 226 is elastically restored, and the positioning portion 102 is pushed to reset.

The edge of the sealing plate 226 may be sealed and fixed with the circumferential side of the movable port 214 corresponding to the mounting pad 212 by means of injection molding, gluing or welding.

By connecting the edge of the sealing plate 226 with the mounting pad 212 to form an integral structure, the movable port 214 is sealed. When the charging interface 100 is installed on the casing 156 of the therapy device, the external moisture or dust is prevented from entering the casing 156 from the movable port 214 and the electronic components such as the circuit board 106 in the casing 156 are prevented from being damaged, and the sealing property of the casing 156 is improved. The elastic reset piece 224 not only drives the positioning portion 102 to automatically reset, but also seals the movable port 214, and eliminates the need to adopt other parts to seal the movable port 214. This simplifies the structure of the charging interface.

By clamping the convex 234 into the clamping bit 218, the edge of the sealing plate 226 is stably connected with the mounting pad 212.

The sealing plate 226 may have a protrusion 232 that protrudes towards the positioning portion 102. The protrusion 232 corresponds to the positioning portion 102, and the protrusion 232 is matched with the movement of the positioning portion 102 to produce elastic deformation.

The mounting pad 212 may comprise a plurality of clamping bit 218 arranged along the circumferential interval of the mounting pad 212. The elastic reset piece 224 may have a plurality of convex 234. Each of the plurality of convex 234 is buckled into each of the plurality of clamping bit 218. This strengthens the stability of the connection between the elastic reset piece 224 and the mounting pad 212. For illustration, the number of clamping bit 218 is four, and the four clamping bit 218 are located around the mounting pad 212. The number of the convex 234 is also four, the four convex 234 are located around the edge of the sealing plate 226. The four convex 234 are buckled into the four clamping bit 218, and the four surrounding areas of the elastic reset piece 224 are connected with the periphery of the mounting pad 212. The stability of the connection between the elastic reset piece 224 and the mounting pad 212 is effectively improved so that the sealing plate 226 can stably produce elastic deformation. This improves the stability of the motion of positioning portion 102.

Referring to FIG. 5 and FIG. 6, the positioning portion 102 is provided with a boss 208 for pushing the switch piece 250. The boss 208 is made to approach and trigger the switch piece 250 by pressing the positioning portion 102. The sealing plate 226 is provided with a first through hole 228, and the boss 208 is penetrated through the first through hole 228.

In another embodiment of the present invention, the positioning portion is slidable over the device casing. The positioning portion may have a protrusion at one side, so that when a user slides the positioning portion over the casing, the protrusion presses the switch piece to form a conductive loop between the circuit board and one or more electro-stimulation element. When the positioning portion is slided towards other side, the protrusion releases the switch pieces and the loop between the circuit board and the one or more electro-stimulation element is released.

The charging pins 104 may be a pogo pin. The charging pins 104 can produce elastic expansion and contraction deformation under force. The sealing plate 226 is provided with a second through hole 230, and the charging pins 104 is penetrated through the second through hole 230. One end of the charging pins 104 is connected to the circuit board 106 by penetrating a second through hole 230 and extending out of the second surface 204. The switch piece 250 is an elastic telescopic structure, so that when the switch piece 250 is pressed through the positioning portion 102, the switch piece 250 is triggered, and the elastic compression deformation is produced. The charging pins 104 also produces elastic compression deformation. When the positioning portion 102 is reset, the switch piece 250 and the charging pins 104 produce elastic elongation deformation, so that the charging pins 104 can maintain a stable contact electrical connection with the circuit board 106, and no additional wire bonding wire is required. It is beneficial to simplify the assembly process and reduce costs.

The sealing plate 226 and the charging pins 104 are elastic. When the pressing force on the positioning portion 102 is revoked, the positioning portion 102 is quickly and stably reset under the elastic force so that that the sealing plate 226 and the charging pins 104 match.

Referring to FIG. 5 and FIG. 6, the charging pins 104 comprise two conductive parts, the two conductive parts respectively located at the opposite two sides of the convex column. One of the charging pins is a positive electrode, and the other is a negative electrode. The first positioning hole 110 and the second positioning hole 112 correspond to the conductive part. The second through holes 230 are two in number and arranged corresponding to the first positioning hole 110 and the second positioning hole 112. The two conductive parts of the charging pins 104 are respectively provided with first positioning hole 110 and the second positioning hole 112 and the corresponding second through holes 230.

In an embodiment, the elastic reset piece 224 can also be an elastic component such as a spring or a shrapnel.

Referring to FIG. 6 and FIG. 8, the mounting pad 212 is provided with two elastic arms 222 for supporting the opposite two sides of the positioning portion 102. The two elastic arms 222 are respectively arranged on the opposite two sides of the movable port 214. The elastic arm 222 may be provided with a clamping bit 218.

When pressing the positioning portion 102 to trigger the switch piece 250, the two elastic arms 222 are respectively supported on the opposite two sides of the positioning portion 102 to bear the partial pressing pressure of the positioning portion 102, so that the elastic reset piece 224 is protected. The elastic reset piece 224 is effectively prevented from being crushed by the stress transition, or the edge of the sealing plate 226 and the mounting pad 212 are separated from each other, and the sealing of the sealing plate 226 to the movable port 214 is invalidated.

Referring to FIG. 5 and FIG. 6, the charging pins 104 is a pogo pin. The pogo pin can be used to support the circuit board, so that the connection conduction of the circuit can be easily realized. The pogo pin can be used to provide a certain elastic force to promote the reset of the positioning portion 102 and improve the pressing feel. When two pogo pins are positioned at the two sides of the boss 208 respectively, the stability of the circuit connection is ensured when the positioning portion 102 is pressed.

Referring to FIG. 5 and FIG. 6, the charging interface 100 further comprises a magnetic suction piece 236 arranged on the positioning portion 102. The magnetic suction piece 236 may be arranged on the side where the second surface 204 is located or embedded in the positioning portion 102.

The magnetic suction piece 236 is used for magnetic adsorption with one end of charging cable, effectively guaranty the stability of electrical contact between charging cable and charging pins 104 to realize stable charging.

The magnetic suction piece 236 can be a magnet or a metal object that can attached to the magnet on the charging cable, such as iron, nickel or cobalt.

In a specific embodiment, the magnetic suction pieces 236 are magnets and the number of magnetic suction pieces 236 is two that are arranged at intervals. When charging, the two magnets on the charging line coupled with the two magnetic suction piece 236 respectively, so that the positive electrode contact on the charging line is accurately in contact with the positive electrode charging pins 104, and the negative electrode contact on the charging line is accurately in contact with the negative electrode charging pins 104.

Referring to FIG. 5, the positioning portion 102 is provided with a slot 210. The slot 210 penetrates through the second surface 204, and the magnetic suction piece 236 is clamped in the slot 210. By arranging the magnetic suction piece 236 in the slot 210, the magnetic suction piece 236 is stably assembled on the positioning portion 102, and the assembly can be done simply and efficiently. The card slots are two in number to accommodate two magnetic suction piece 236 to provide stability in assembling magnetic suction piece 236.

In an embodiment of the present invention, one or more positioning holes comprises a USB port having one or more charging pins for charging the device.

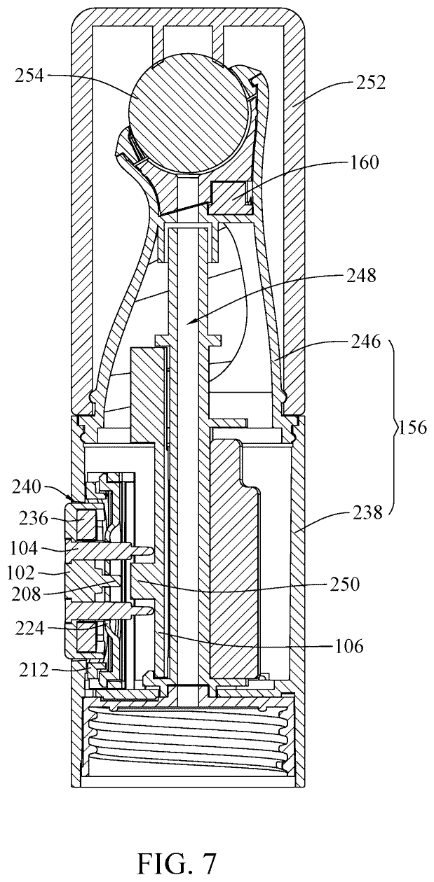

Referring to FIG. 1, FIG. 7 and FIG. 8, the charging interface is used for charging a therapy device. The therapy device comprises a casing 156, a circuit board 106, a switch piece 250, an output device and the charging interface 100. The circuit board 106 is arranged in the casing 156, the switch piece 250 is arranged on the circuit board 106, and the output member is arranged on the casing 156. The output part is electrically connected with the circuit board 106, the positioning portion 102 of the charging interface 100 is arranged on the casing 156 and corresponds to the switch piece 250. The charging pins 104 is electrically connected with the circuit board 106.

The hand-held therapy device may comprise a bottle, a cap to hold the bottle, a liquid head to hold the bottle, a scoop for skin care products, one or more electro-stimulation element to provide alternate therapy to the user, a light therapy instrument, the introduction device or the skin beauty device that can be used alone. The electro-stimulation element is used for generating therapeutic light, micro-current, vibration, cooling or heat, and the electro-stimulation element for performing an alternate therapy during application of a cosmetic product. The one or more electro-stimulation elements may include different types of luminaires that emit red, ultraviolet, visible, and other light to perform phototherapy of the skin. The electro-stimulation element may also include a micro-current generator to generate micro-current. The electro-stimulation element may also include a heating sheet, which produces and transmits heat. The electro-stimulation element may also include a cooling sheet, which can be used to cool a surface. The electro-stimulation element may also include a vibrator to provide vibration effect during massaging. The one or more electro-stimulation element is selected from a group consisting of Light Emitting Diodes (LEDs), lasers, heating elements, cooling elements, vibration elements, electrodes, micro-current element and combinations thereof.

The circuit board 106 is used to drive the electro-stimulation element, and the electro-stimulation element is used to carry out beauty therapy. The switch piece 250 is triggered by pressing the positioning portion 102, and the switch piece 250 is opened or closed by pressing the positioning portion 102, so that the operation of electro-stimulation element can be controlled.

The switch piece 250 may be a push-type mechanical switch, so that the switch piece 250 can be controlled to open or close by pressing the positioning portion 102. The switch piece 250 can also be a mechanical switch or a tactile switch.

A conductive plate may be arranged at the position corresponding to the charging pins 104 on the circuit board 106. The charging pins 104 is matched against the conductive disk, and the boss 208 is matched with the switch piece 250. This eliminates the need for welding, and the positioning portion 102 can be driven to reset by utilizing a pogo pin. Two pogo pins are positioned at the opposite two sides of switch piece 250 respectively, to improve the stability of positioning portion 102.

The output may include one or more electro-stimulation element consisting of phototherapy lamps, heating parts, vibrating parts and refrigerating parts. The electro-stimulation element includes but is not limited to a stimulation element such as Light Emitting Diodes (LEDs), lasers, heating elements, cooling elements, vibration elements, electrodes, micro-current element, an actuator, a pump, a motor, a vibrator, a heater, a piezoelectric element, an abrasive element.

The light emitted by the light therapy lamp is irradiated to the user's skin, which is used for phototherapy on the user's skin, promoting blood circulation and strengthening cell metabolism, accelerating the absorption of skin care products by the skin, and healing acne, beauty and other effects. The heating part is energized to generate heat, and the heat is transferred to the skin, which increases the temperature of the skin, promotes the opening of pores, and facilitates the absorption of skin care products by the skin. The vibrating parts are energized to produce vibrations and act on the user's skin, helping to soothe skin tension, promote skin detoxification, and reduce puffiness and fine lines. Refrigerants are energized and applied to the user's skin to lower temperatures, soothe the skin, reduce swelling or relieve pain.

The hand-held therapy device adopts the charging interface 100 having the charging pins 104 penetrated through the first positioning hole 110 and the second positioning hole 112. The charging pins 104 is integrated on the positioning portion 102, so that the charging pins 104 and the positioning portion 102 form a single unit, so that only the hole position of assembling the positioning portion 102 is reserved on the casing 156 of the therapy device. The number of hole positions on the casing 156 is conducive to reducing the number of holes on the casing 156, and the sealing property of the casing 156 is improved. By integrating the charging pins 104 on the positioning portion 102, the charging pins 104 does not need to occupy the extra area of the outer surface of the casing 156 that effectively reduces the occupied area of the outer surface of the casing 156, and makes the appearance of the therapy device neat and aesthetically better.

In an embodiment of the present invention, the charging interface can be integrated into a phototherapy face mask. The phototherapy mask comprise a backing layer, an electronic layer comprising a layer having one or more electro-stimulation element, a skin contacting layer and a fastening means. The electronic layer comprises a main circuit board connected with one or more electro-stimulation element. The electronic layer is housed in a casing. The charging interface having positioning portion is mounted on the casing of the phototherapy mask. This will make the phototherapy face mask aesthetically pleasing and reduce the number of holes needed for mounting charging interface and the switch, which helps in making the mask waterproof.

Referring to FIG. 7 and FIG. 8, the casing 156 is provided with an opening 240 arranged on the circumferential side wall of the casing 156. The mounting pad 212 is arranged in the casing 156 and corresponds to the position of the circuit board 106. The switch piece 250 is arranged on one side of the circuit board 106 towards the mounting pad 212, and the movable port 214 of the mounting pad 212 is arranged corresponding to the opening 240. One side of the positioning portion 102 with the first surface 202 protruding from the opening 240. The charging interface 100 is arranged on the circumferential side wall of the casing 156, and the positioning portion 102 can be conveniently pressed. When the charging is needed, the charging cable can be conveniently connected to the charging pins 104 electrically.

The casing 156 may have two mounting strip 242 arranged and extend along the length direction of the casing 156. The two mounting strip 242 are located on opposite sides of the opening 240. The two mounting strip 242 form two mounting groove 244 through the inner periphery wall surface of the casing 156. The opposite two sides edges of the mounting pad 212 are respectively clamped in the two mounting groove 244, so that the mounting pad 212 is assembled in the casing 156.

Referring to FIG. 5, FIG. 6 and FIG. 8, the mounting pad 212 is provided with a stop block 220. The stop block 220 is arranged at one end of mounting pad 212 along the length direction of casing 156. When mounting pad 212 is installed on the casing 156, along the length direction of the casing 156, the mounting pad 212 slides into the mounting groove 244, until the stop block 220 is butted against one end of the mounting strip 242.

Referring to FIG. 6 and FIG. 7, the specific steps for assembling the charging interface 100 to the casing 156 involves assembling a positioning portion 102, a charging pins 104, a mounting pad 212 and an elastic reset piece 224 to obtain a charging interface 100. In the first step, the charging pins 104 is assembled with the positioning portion 102 to obtain a first half assembly. The charging pins 104 is penetrated into the first positioning hole 110 and the second positioning hole 112, and the charging pins 104 is sealed between the first positioning hole 110 and the second positioning hole 112, using a filling sealant, sealing strip, etc. Assembling the elastic reset piece 224 with the mounting pad 212 to obtain a second half assembly. For example, the convex 234 is buckled into the clamping bit 218, and the first through hole 228 and the second through hole 230 on the elastic reset piece 224 are correspondingly arranged with the movable port 214 on the mounting pad 212. The method may optionally include assembling the magnetic suction piece 236 to the positioning portion 102. For example, the magnetic suction piece 236 is set into the slot 210. The next step is assembling the first half assembly with the second half assembly to obtain a charging interface 100. For example, the positioning portion 102 is movably worn in the movable port 214, the limiting portion 216 is movably snapped into the limiting groove 206, the boss 208 is penetrated through the first through hole 228, and the charging pins 104 is penetrated through the second through hole 230. The charging interface 100 is assembled with the casing 156. For example, along the length direction of the casing 156, the mounting pad 212 is slid into the mounting groove 244 until the stop block 220 is butted against one end of the mounting strip 242, and the inner periphery wall surface of the mounting pad 212 and the casing 156 is sealed, or the mounting pad 212 of the charging interface 100 is injected into one with the casing 156.

In one embodiment of the present application, the therapy device comprises a bottle body, the shell is connected with one end of the bottle body with a bottle mouth, and a liquid discharge channel that is communicated with the opening of the bottle body is formed in the shell.

The bottle body can be used to contain cosmetics, such as creams, liquids, etc., in which creams can be face creams, eye creams, body creams, etc., and liquid medicines can be essences, lotions, essential oils, etc. The output part is located at one end of the shell, and the other end of the shell is connected with the bottle mouth. The bottle mouth is sealed to protect the skin care products in the bottle body.

Referring to FIG. 7, the casing 156 comprises an assembly section 238 and an export section 246. The assembly section 238 is connected with one end of the bottle body with a bottle mouth. The charging interface 100 is arranged at the assembly section 238. The output part 160 is arranged at one end of the export section 246 far away from the assembly section 238. The liquid medicine in the bottle body is output at one end of the export section 246 far away from the assembly section 238 through the liquid outlet channel 248. The therapy device may further comprises an outer cover 252. The outer cover 252 covers the export section 246 and is used to protect the export section 246 and prevent the export section 246 from being polluted and damaged.

The therapy device may further comprises a coating part 254. The coating part 254 is arranged at one end of the export section 246 away from the assembly section 238. The coating part 254 is used for applying the medicinal solution output at one end of the export section 246 away from the assembly section 238 to the skin and massaging the skin. By using the coating part 254, it is possible to evenly apply the liquid medicine to the skin when the liquid is out, and massage the skin to play the role of beauty massage. The coating part may be a ball and the rolling arrangement is arranged at one end of the export section 246, which is far away from the assembly section 238. The liquid medicine is applied to the skin through the coating part 254, and the skin is rolled and massaged, and the resistance is small, and is convenient to use.

In an embodiment of the present invention, providing a single unit for charging as well as controlling the electro-stimulation element on a therapy device provides various advantages. Providing a single unit for charging interface and controlling one or more electro-stimulation element needs only a single mounting hole on the device casing instead of two separate mounting holes. This is conducive for providing waterproofing ability such that provisions are made in one place instead of two. Moreover, with positioning portion and switch at one place, the aesthetic look of the therapy device improves. Another advantage of providing switch on the charging interface is that it avoids accidental switching on the switching device when the charging interface is connected to the charging controller. Further it improves the device's battery life as it avoids parallel use of device along with the charging of the device.

In an embodiment of the present invention, a charging connector or charging cable or charging dock is provided. The charging connector connects with the charging interface on the therapy device to electrically charge the therapy device. The charging connector can be a male charging connector or a female charging connector. When in use, the male charging connector is arranged on the power output device and connected to the charging interface on the therapy device, so that on docking the transmission of power takes place. The charging connector comprises a shell, an electrical connection assembly and a magnetic suction piece. The magnetic suction piece is located in the housing and/or in the charging terminal. The magnetic suction piece's magnetic attraction effect is used to improve the contact stability between the charging connector and the charging interface.

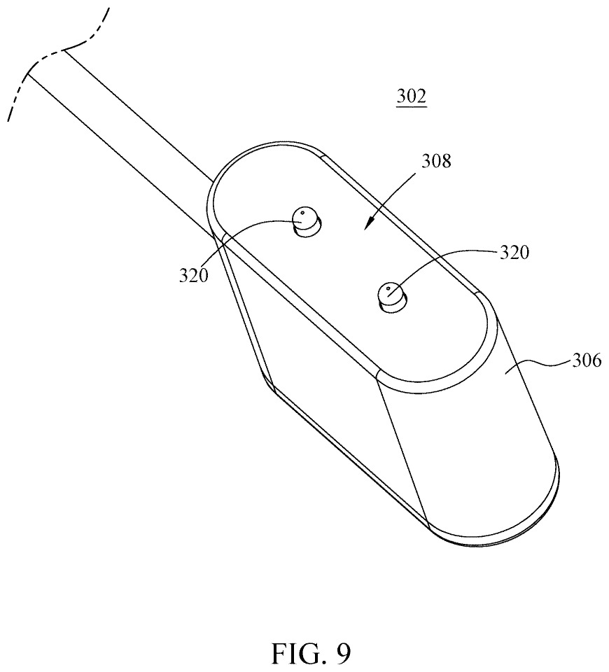

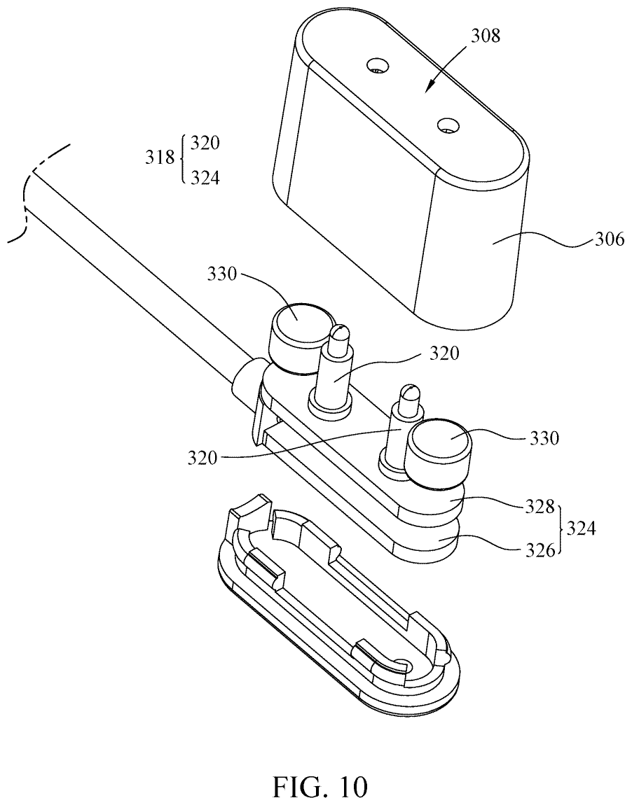

Referring to FIG. 9 and FIG. 10, the charging connector 302 comprises a shell 306, an electrical connection assembly 318 and a magnetic suction structure 330. The shell 306 is the main bearing structure, which is used for installing the electrical connection assembly 318 and the magnetic suction structure 330. The shape and structure of the shell 306 can be but not limited to a cylindrical shell, a square shell, etc.

The electrical connection assembly 318 is a signal or current transmission component in the charging connector 302. The electrical connection assembly 318 comprises a charging terminal 320 having one end arranged in the shell 306 and the other end arranged through the shell 306 to the outside. The charging terminal 320 can be a male charging terminal or a female charging terminal.

The magnetic suction structure 330 is a permanent magnet with magnetism, for example, the magnetic suction structure 330 can be a permanent magnet prepared by iron, cobalt, nickel and the like. Alternatively, the magnetic suction structure 330 can also be a magnet with magnetic properties, for example, the magnetic suction structure 330 can be a coil that supplies an electric current, or a ring structure, etc.

The shape of the magnetic suction structure 330 is not limited, and can be adjusted according to the actual use case, for example, the magnetic suction structure 330 can be a cylindrical magnetic suction piece, a cubic magnetic suction piece, a layered magnetic suction piece or a sheet magnetic suction piece.

The magnetic suction structure 330 is arranged at the shell 306 and/or the charging terminal 320 or on both the shell 306 and the charging terminal 320. For example, the magnetic suction structure 330 is a columnar permanent magnet, and the columnar permanent magnet can be arranged in the shell 306 and fixed on the inner side wall of the shell 306. Alternatively, the columnar permanent magnet can be arranged outside the shell 306 and fixed on the outer side wall of the shell 306. Alternatively, columnar permanent magnets are arranged on both the inner and outer side walls of the shell 306. Alternatively, the magnetic suction structure 330 is a magnetic coating layer coated on the outer surface of the charging terminal 320.

When the charging connector 302 is electrically connected and adapted, the connection stability of the charging terminal 320 can be improved through the magnetic suction structure 330 on the shell 306 to attract each other magnetically. Alternatively, the magnetic suction structure 330 on the charging terminal 320 can also be magnetically attracted to each other to improve the connection stability of the charging terminal 320. Alternatively, a magnetic suction structure 330 can be arranged on both the shell 306 and the charging terminal 320 to further improve the connection stability of the charging terminal 320 after the electrical connection.

In an embodiment of the present invention, the charging terminal 320 may form either male or female type connector. The male or female type charging connector depends on the corresponding type of charging interface on the device.

Referring to FIG. 9 to FIG. 11, the shell 306 has a first end plate 308. The first end plate 308 is used for exposing the charging terminal 320 to the outside, and the magnetic suction structure 330 is arranged on the first end plate 308.

The first end plate 308 is part of the shell 306 that will be directly butted to contact when the charging connector 302 connects to the charging interface. The charging terminal 320 needs to be threaded through the first end plate 308 and to the outside.

The magnetic suction structure 330 is arranged on the first end plate 308, and the magnetic suction distance can be further shortened to improve the magnetic attraction force between the charging connector 302 and the charging interface.

The shell 306 is also provided with other end plates, and the first end plate 308 is enclosed with other end plates to form a hollow structure with an accommodating cavity inside to install the electrical connection assembly 318 and other components.

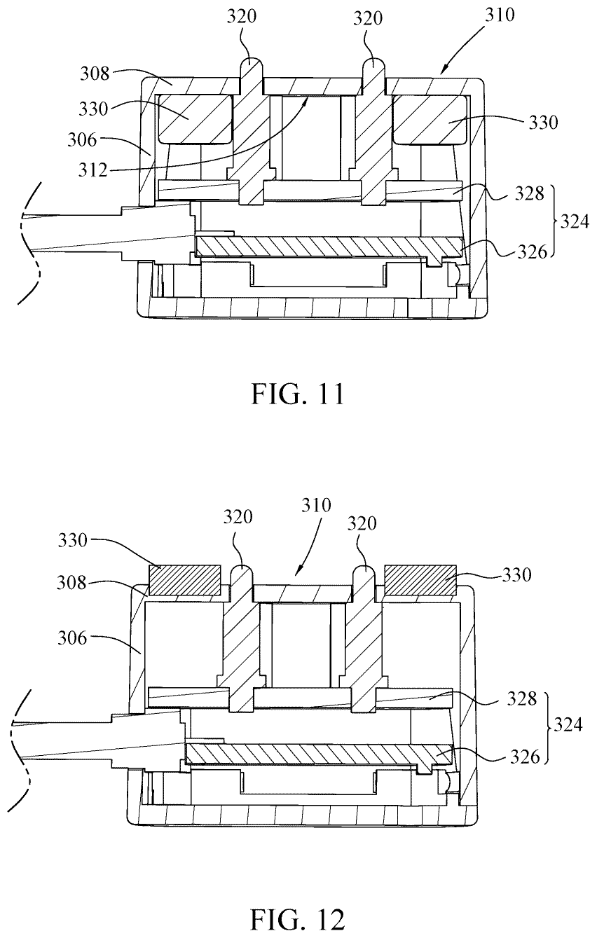

Referring to FIG. 11 to FIG. 13, the first end plate 308 has an outer surface 310 and an inner surface 312 arranged relative to the outer surface 310, and the magnetic suction structure 330 is arranged on the outer surface 310 and/or the inner surface 312.

The outer surface 310 is the surface of the first end plate 308 facing outward, that is, the surface where the charging connector 302 is in contact with the charging interface, and the inner surface 312 is opposite to the outer surface 310 and is located at the surface inside the shell 306.

The magnetic suction structure 330 can be arranged on the outer surface 310 of the first end plate 308. Alternatively, it can also be arranged on the inner surface 312 of the first end plate 308. Alternatively, the magnetic suction structure 330 can also be arranged on the outer surface 310 and the inner surface 312 of the first end plate 308.

The connection mode between the magnetic suction structure 330 and the outer surface 310 and/or the inner surface 312 includes, but is limited to, plugging, bonding and threaded connection.

A retaining structure can be formed by convex extension on the inner surface 312 of the first end plate 308, and a magnetic suction structure 330 is arranged in the enclosure structure. Alternatively, a mounting groove may be formed in the inner surface 312 of the first end plate 308 to arrange the magnetic suction structure 330 in the mounting groove. In the same way, an enclosure structure or a mounting groove can also be arranged on the outer surface of the first end plate 308 to fix the magnetic suction structure 330.

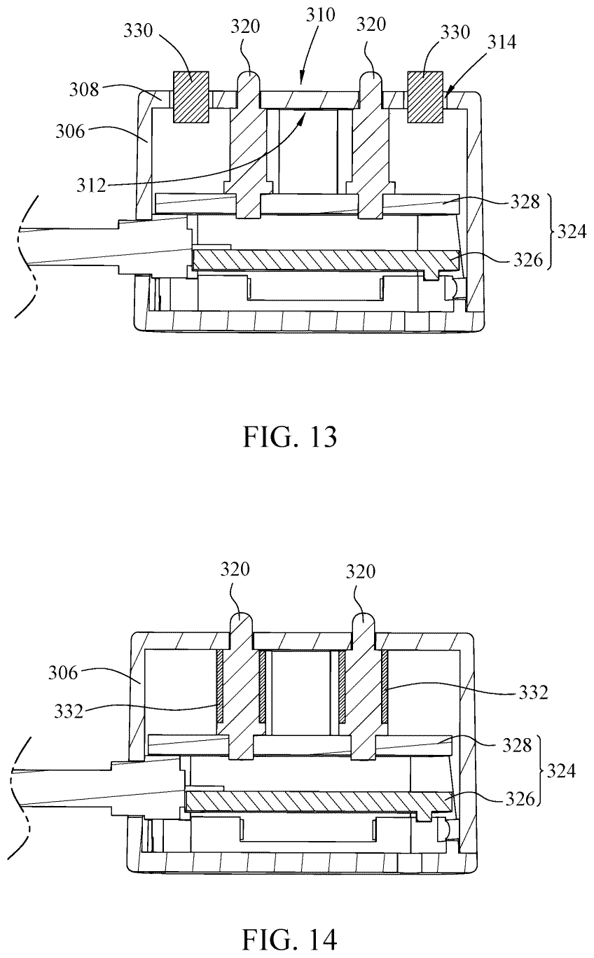

Referring to FIG. 13, the first end plate 308 is provided with a through hole 314 penetrating the outer surface 310 and the inner surface 312, and the magnetic suction structure 330 is placed in the through hole 314. The through hole 314 is a hole structure that penetrates through the first end plate 308 and makes the shell 306 communicate inside and outside. The hole structure of the through hole 314 includes but is not limited to straight holes, step holes, oblique holes, etc., and the hole shapes of the through holes include but are not limited to circular through holes, square through holes, oval through holes, etc. The magnetic suction structure 330 can be fixed through the through hole 314 to shorten the magnetic suction distance.

The magnetic suction structure 330 is placed in the through hole 314, and the magnetic end face of the magnetic suction structure 330 is flush with the outer surface 310, so that when the charging connector 302 connects with the charging interface of the therapy device, the corresponding magnetic suction structure 330 can directly contact and magnetically attract each other, so as to improve the stability.

The profile of the first end plate 308 is circular, oval, gourd-shaped or runway-shaped. The profile of the first end plate 308 refers to the shape formed by the projection of the first end plate 308 in the direction of projection perpendicular to the outer surface 310. The outline of the first end plate 308 is set to circular, oval, hoist-shaped or runway-shaped. The contact area of the shell 306 of the charging connector 302 during electrical connection can be effectively reduced.

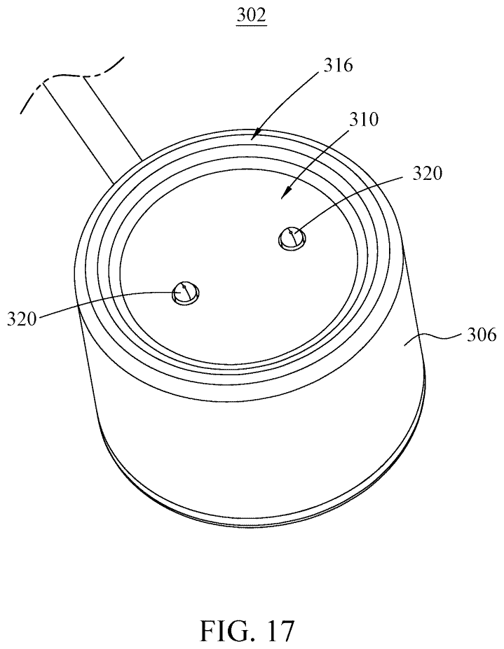

As shown in FIG. 17, the shell 306 is a cylindrical structure, and the outline of the first end plate 308 is circular. The outer diameter of the shell 306 is consistent, so that the overall volume of the shell 306 is smaller.

As shown in FIG. 9, the shell 306 is a cylindrical structure, and the outline of the first end plate 308 is runway-shaped. The outer diameter of the shell 306 is the same, so that the overall volume of the shell 306 is smaller.

The shell 306 can also be columnar, but the outer diameter of the shell 306 can be adjusted. For example, from the plugging assembly direction of the deviated charging connector 302, the outer diameter of the shell 306 is gradually increasing from the first end plate 308, so that the one end is large, and the other end is small.

Referring to FIG. 17, a positioning structure 316 is formed on the outer surface 310, and the positioning structure 316 surrounds the charging terminal 320. The positioning structure 316 is a structure formed on the outer surface 310 and is used for the positioning the charging interface docking on the charging terminal 320. The positioning structure 316 includes, but is not limited to, grooves, protrusions, and combinations of grooves and protrusions formed on the outer surface 310. The positioning structure 316 is around the periphery of the charging terminal 320, and play a corresponding positioning role for the butt connection of the charging terminal 320. The positioning structure 316 also avoid the short circuit when the charging terminal 320 gets dislocated. The positioning structure 316 formed on the outer surface 310 is an annular protrusion bar surrounding the periphery of the charging terminal 320, which forms a butt adaptation relationship with the groove of the shell of the corresponding charging connector.

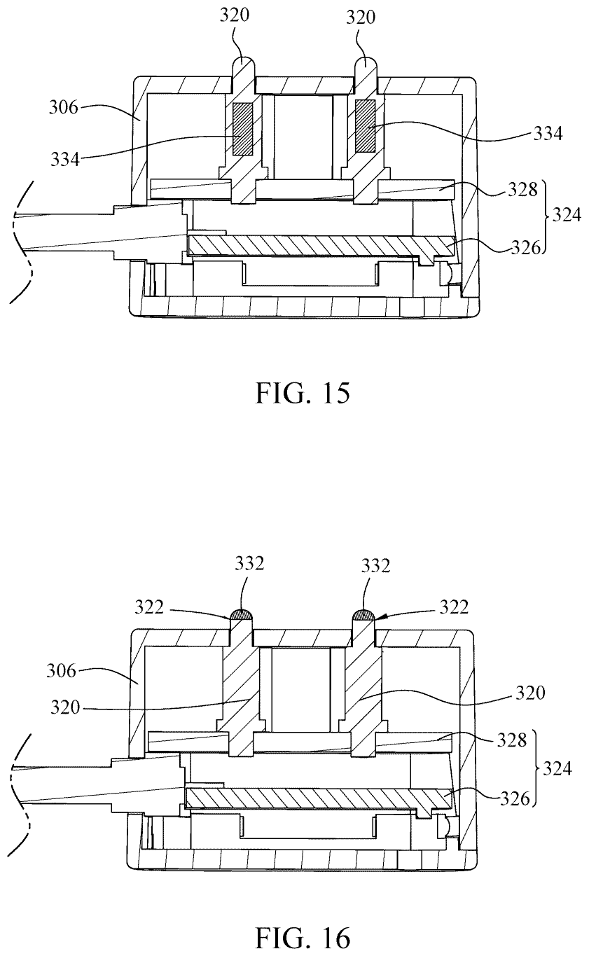

Referring to FIG. 14 to FIG. 16, the magnetic suction structure 330 is arranged on the charging terminal 320. When the charging connector 302 is butted with the charging interface and connected, the charging terminals are docked.

The magnetic suction structure 330 is set on the electrical connection end, or the magnetic suction structure 330 can be arranged on the outer surface 310 of the charging terminal 320. The magnetic suction structure 330 can also be arranged inside the charging terminal 320. Alternatively, the magnetic suction structure 330 may also be arranged inside the charging terminal 320, and some parts extend to the outer surface 310 of the charging terminal 320, or extend to the outside of the charging terminal 320.

For example, the magnetic suction structure 330 is a magnetic coating coated on the outer surface 310 of the charging terminal 320. The magnetic coating can be coated with the entire outer surface 310 or the partial outer surface 310 of the charging terminal 320. The magnetic coating is also conductive, and the corresponding charging terminal 320 conduct electricity when plugged in.

For example, the magnetic suction structure 330 is a permanent magnet block, and the permanent magnet block is arranged in the charging terminal 320, so that the electrical terminal provides a corresponding magnetic attraction force when docking.

Referring to FIG. 14 to FIG. 16, the magnetic suction structure 330 comprises a magnetic layer 332 coated on the outside of the charging terminal 320, or the magnetic suction structure 330 comprises a magnetic body 334 arranged in an charging terminal 320. The magnetic layer 332 is a magnetic suction structure 330 with a thin thickness, for example, the magnetic layer 332 can be a coating with a magnetic substance, a sputtering layer, etc., or the magnetic layer 332 can also be a magnetic sheet with a thin thickness. The magnetic body 334 is a block permanent magnet with a certain volume.

The magnetic layer 332 can be formed on the whole outer wall or part of the outer wall of the charging terminal 320 by spraying, sputtering, electroplating, etc. In another example, the magnetic body 334 can be manufactured synchronously with the charging terminal 320 by means of integral molding, and the magnetic body 334 can be fixed in the charging terminal 320 after the manufacturing is completed, and the mounting hole and other structures can be formed by milling and other means.

Referring to FIG. 16, the charging terminal 320 has a plugging end face 322, and the plugging end face 322 is coated with a magnetic layer 332. Alternatively, the magnetic body 334 is arranged in the charging terminal 320 and is close to the plugging end face 322.

The plugging end face 322 is the end face of the charging terminal 320 that is directly in contact when it is connected to each other, and the plug-in end can be a plane, an arc surface or a waveform surface, etc., or the plugging end face 322 can also be made up of a plurality of sub-surfaces.

In this way, a magnetic layer 332 is formed on the plugging end face 322, which is more conducive to providing the connection stability of the two charging terminals 320 when docking.

Alternatively, the magnetic body 334 can be set close to the plugging end face 322 to provide the corresponding magnetic force.

In some embodiments, the shell 306 has a second end plate (not shown in the FIG.) arranged opposite to the first end plate 308, an indicator light (not shown in the FIG.) is arranged on the second end plate, and the indicator light is electrically connected with the charging terminal 320.

The second end plate is a board structure that is opposite to the first end plate 308. The second end plate should be a board structure in which the charging connector 302 faces the outside or faces the user after docking.

Thus, an indicator light is arranged on the second end plate is to provide a prompt indication after the charging terminal 320 completes docking, and the indicator light is installed on the charging connector 302. The volume of the electrical connector that is docked with the charging connector 302 can be effectively reduced, and the difficulty of installing the indicator light on the space of the electrical connector of the docking is reduced.

Referring to FIG. 10, the electrical connection assembly 318 further comprises a control board group 324 arranged in the shell 306. The control board group 324 comprises a substrate 326 and a supporting plate 328 arranged at intervals along the height direction of the shell 306. The substrate 326 is electrically connected with the supporting plate 328, and one end of the charging terminal 320 is arranged on the supporting plate 328 and is electrically connected with the supporting plate 328.

The substrate 326 is the main control board and is used for processing the corresponding electrical signal, the supporting plate 328 is used for bearing and fixing the charging terminal 320. The substrate 326 and the supporting plate 328 can form a position relationship between each other through support columns such as columns, and can also form a position relationship with the inner wall of the shell 306 that is spaced apart.

Optionally, in the height direction of the shell 306, the projection of the supporting plate 328 falls into the projection of the substrate 326, that is, the size of the supporting plate 328 is less than or equal to the size of the substrate 326, so that the cross-sectional area of the shell 306 in the perpendicular height direction can be smaller. The ratio of the square of the height of the shell 306 to the cross-sectional area is larger, and the overall structure of the shell 306 presents the structural characteristics of “long and thin”.

Compared with the charging connector 302 that adopts a single control board, the substrate 326 and the supporting plate 328 are stacked in layers, the cross-sectional area of the shell 306 can be further reduced, and the shell 306 is more conducive to presenting the structural characteristics of “long and slender”, so that the charging connector 302 can adapt to some use scenarios with small butt contact area.

As shown in FIG. 10, in the height direction of the shell 306, the projection of the substrate 326 and the supporting plate 328 are both runway shaped, and the cross-sectional shape of the shell 306 is adapted to the substrate 326 and the supporting plate 328 is also runway shaped, so that the overall shape of the shell 306 presents the structural characteristics of high and thin.

For example, in the height direction of the shell 306, the projections of the substrate 326 and the supporting plate 328 are circular, and the cross-sectional shape of the shell 306 is adapted to the substrate 326 and the supporting plate 328 is also circular, so that the overall shape of the shell 306 presents a long and slender structural characteristic.

In an embodiment of the present invention, the charging connector configuration is provided so that the charging dock can be adapted to different type of therapy device. The shell 306 is provided with a recessed portion 404 formed on the outer side of the shell 306. The recessed portion 404 comprises a first groove 406 and a second groove 408. The first groove 406 is a circular groove and the second groove 408 is connected to the periphery of the first groove 406.

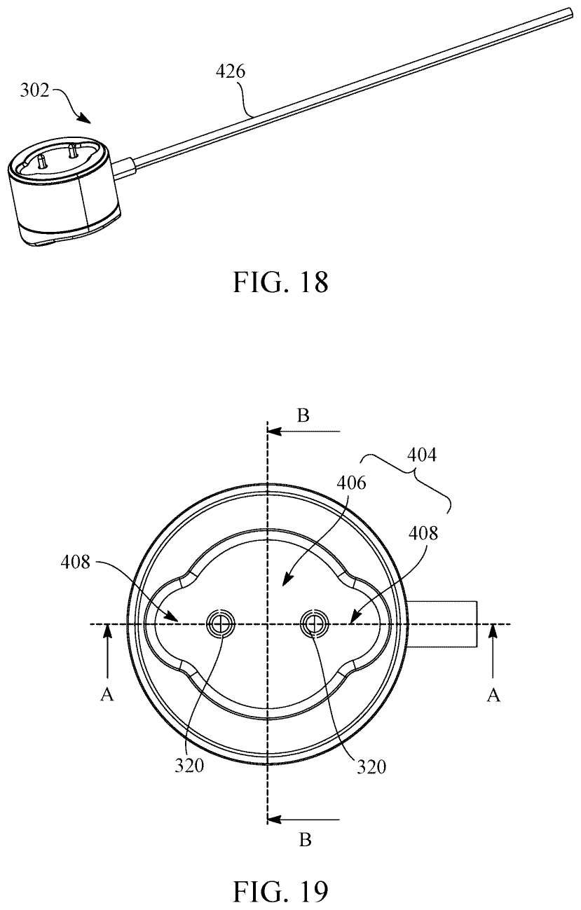

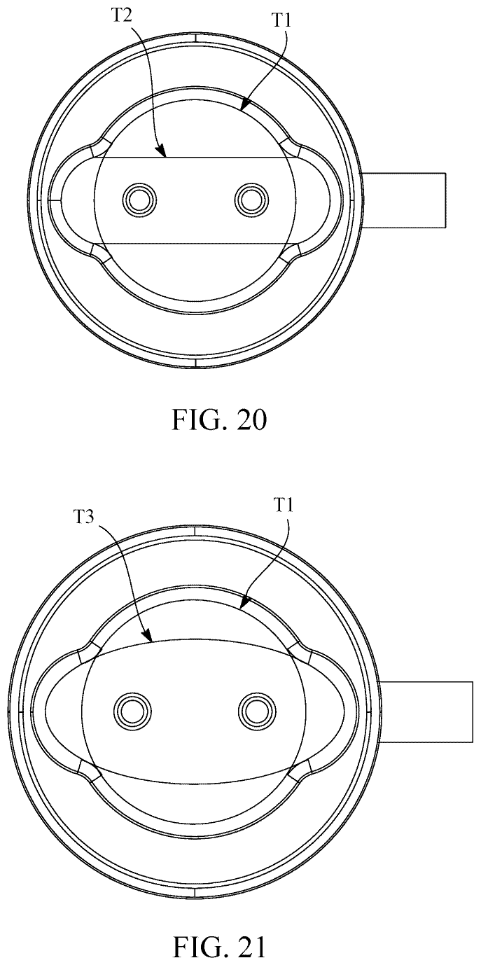

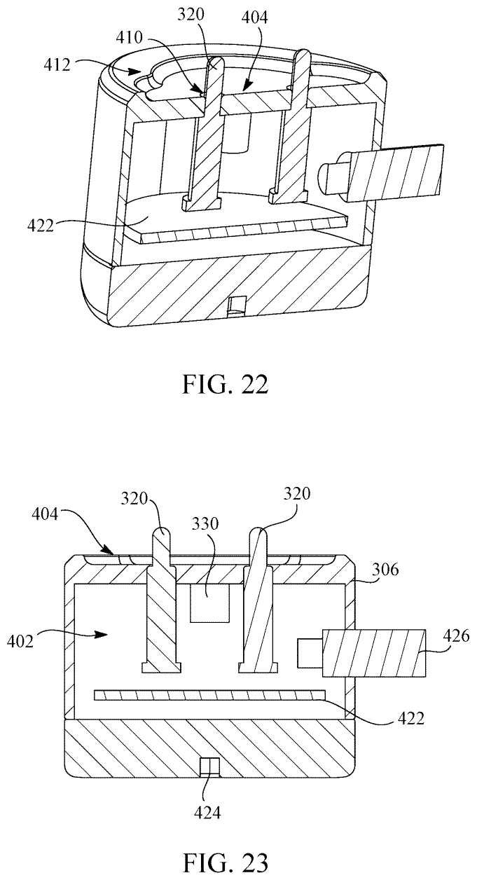

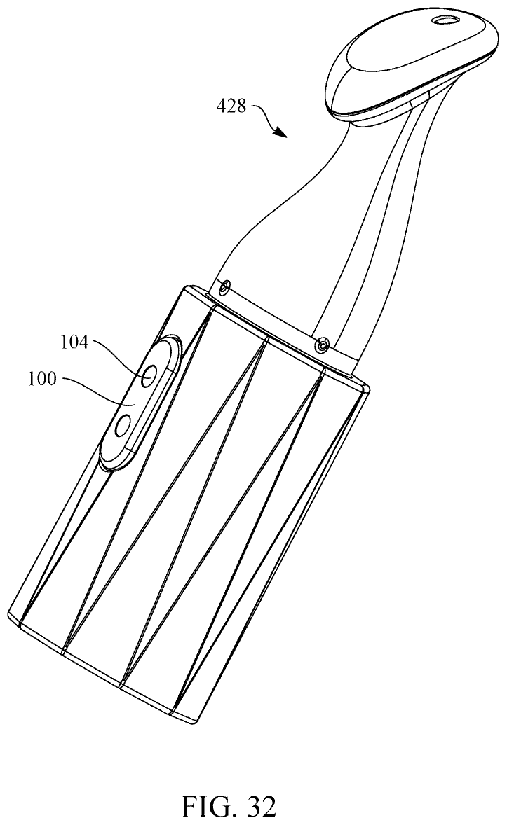

Referring to FIG. 18 and FIG. 32, the charging connector 302 comprises the shell 306, two charging terminal 320 and a circuit board 422. The shell 306 has a storage chamber 402, a recessed portion 404 and two mounting holes 410. The recessed portion 404 is formed on the outer side surface of the shell 306. The recessed portion 404 comprises a first groove 406 and a second groove 408 that communicate with each other. The first groove 406 is a circular groove (shown as T1 in FIG. 20 and FIG. 21). The second groove 408 is connected to the periphery of the first groove 406, and the mounting hole 410 penetrates through the first groove 406 and communicates with the storage chamber 402. Two charging terminal 320 are arranged in the two mounting holes 410. The charging terminal 320 are sealed and connected with the hole wall of mounting holes 410. The circuit board 422 is arranged in the storage chamber 402 and is electrically connected with the charging terminal 320.

The charging connector 302 is provided with a first groove 406 and a second groove 408. A separate first groove 406, or a part of the first groove 406 and the second groove 408, or the first groove 406 and the second groove 408 as a whole can be combined to form different shapes respectively, so as to adapt the charging interface 100 of different shapes on the therapy device 428, and to position the charging interface 100 of different shapes. The shape of the first groove 406 and the second groove 408 ensures that the charging pins 104 on the charging interface 100 is aligned with the charging terminal 320 on the charging connector 302 to realize electrical connection. In this way, there is no need to develop different charging connector 302 for the charging interface 100 of different shapes, improve the versatility of charging connector 302, and thereby achieve the purpose of reducing cost.

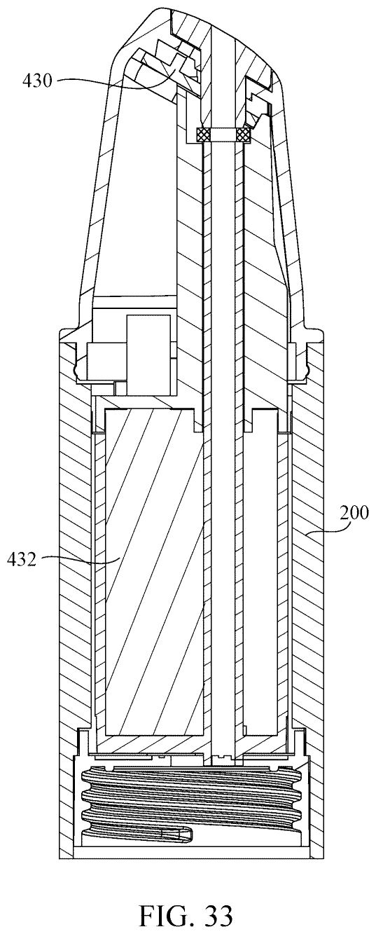

Referring to FIG. 32 and FIG. 33, the therapy device 428 has a charging interface 100. The charging interface 100 protrudes from the outer surface of the casing 200 of the therapy device 428. A charging pins 104 is embedded in the charging interface 100. One end of the charging pins 104 is electrically connected with the battery 432 of the therapy device 428, and the other end of the charging pins 104 is exposed on the outer surface of the charging interface 100. Different styles of therapy devices 428 may have different shapes of charging interface 100, including but not limited to circles, ovals, runway-shapes, triangles, etc.

When the charging connector 302 needs to charge the therapy device 428, and the charging interface 100 of the therapy device 428 is circular, the first groove 406 can be inserted into the circular charging interface 100 and fit with it to realize positioning, and ensure that each terminal is aligned to complete electrical connection during charging. If the charging interface 100 of the therapy device 428 is elongated, a part of the first groove 406 and all of the second groove 408 can be combined to form a strip-shaped groove for the charging interface 100 to be inserted, and the second groove 408 can be completely matched with a part of the charging interface 100 to achieve accurate positioning.

In the embodiment of the present invention, the shape of the second groove 408 includes, but is not limited to, a semicircle, a quarter circle, a triangle, a square, etc. Optionally, the outer contour of the second groove 408 is arc-shaped, so that the second groove 408 as a whole approximates a circle, such as a complete circle or an incomplete circle.

Optionally, the radius of the second groove 408 is smaller than the radius of the first groove 406. When the radius of the second groove 408 is approximately semicircular, a part of the second groove 408 and a part of the first groove 406 can be combined to form a runway-shaped or elliptical-shaped, so that it can be adapted to three different charging interface 100 of runway-shaped, elliptical and circular at the same time.

In some embodiments, the recessed portion 404 comprises two second grooves 408. The two second grooves 408 are symmetrically arranged with the first groove 406 as the center. The center of the two second grooves 408 are collinear with the center of the circle of the first groove 406. The center line of the two mounting holes 410 coincides with the center line of the two second grooves 408. When the outer contour of the second groove 408 is arc-shaped, the center of the second groove 408 refers to the center of the circle. When the outer contour of the second groove 408 is square, the center of the second groove 408 refers to the intersection point of the diagonals.

Referring to FIG. 20 and FIG. 21, two second grooves 408 are connected end-to-end through imaginary lines, and the two second grooves 408 and a part of the first grooves 406 can form the structure of partial strips. Taking the arc-shaped second groove 408 as an example, the two second grooves 408 can be connected with a part of the first groove 406 to form a runway shape as shown in T2 in FIG. 20 or an elliptical shape as shown in T3 in FIG. 21. The two mounting holes 410 are arranged along the center connection line of the two second grooves 408, that is, the two mounting holes 410 will be located on the center line of runway-shaped or elliptical, so that the charging terminal 320 in the mounting hole 410 can be positioned within the range of the charging interface 100, and the butt conduction with the charging pins 104 on the charging interface 100 can be realized. In this way, different styles of therapy devices 428 do not need to change the orientation of their two charging pins 104.

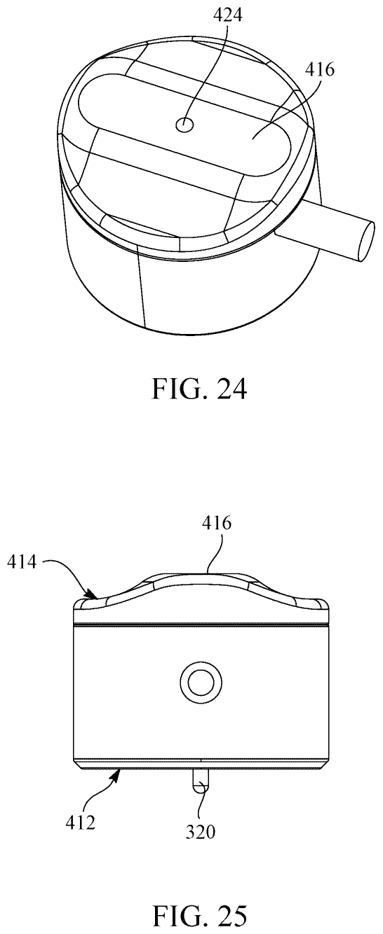

Referring to FIG. 24 and FIG. 25, the shell 306 has a first outer surface 412 and a second outer surface 414. The recessed portion 404 is formed in the first outer surface 412. The second outer surface 414 is provided with an convex part 416. The convex part 416 is elongated, and the length direction of the convex part 416 is parallel to the distribution direction of the first groove 406 and the second groove 408. When the charging interface 100 on the therapy device 428 is positioned by the positioning groove jointly formed by the second groove 408 and a part of the first groove 406, the user does not need to flip the charging connector 302 to observe whether the length direction of the positioning groove is aligned with the charging interface 100. The user can directly observes the length direction of the convex part 416 and the charging interface 100 of the therapy device 428 from the front after holding the charging connector 302, so that the charging interface 100 can be accurately inserted into the positioning groove. The convex part 416 is equivalent to the indication effect of carrying out alignment during charging, and in addition, the convex part 416 can also strengthen the structure of the shell 306.

The convex part 416 is indicated to be connected with the second outer surface 414 by an arc transition, so that a smooth outer surface can be formed.

The two second grooves 408 are connected end-to-end through imaginary lines to form a first figure (as T2 in FIG. 20 or T3 as in FIG. 21). The outer contour of the cross-section indicating the convex part 416 is the same as the outer contour of the first figure, and the cross-section is a cross-section perpendicular to the protruding height direction of the convex part 416. In the embodiment of the present invention, the outer contour of the cross-section indicating the convex part 416 is the same as the outer contour of the first figure, which means that the contour is basically the same, but the size may be different. For example, the first figure may be an ellipse, a runway shape or a rectangle, etc., correspondingly, the outer contour of the cross-section indicating the convex part 416 is an ellipse, a runway shape or a rectangle.

The shape of the convex part 416 and the first figure is basically the same, therefore, the orientation of the first figure can be indicated more intuitively, and the charging interface 100 on the therapy device 428 is aligned and mated with the first figure.

The charging connector 302 further comprises an indicator light 424, and the indicator light 424 is arranged at the convex part 416. Optionally, the indicator light 424 is embedded in the convex part 416. When the charging pins 104 of the therapy device 428 is aligned with the charging terminal 320 of the charging connector 302 and conducted, the indicator light 424 lights up, indicating that it is charging. When charging is completed, the luminous color of indicator light 424 changes.

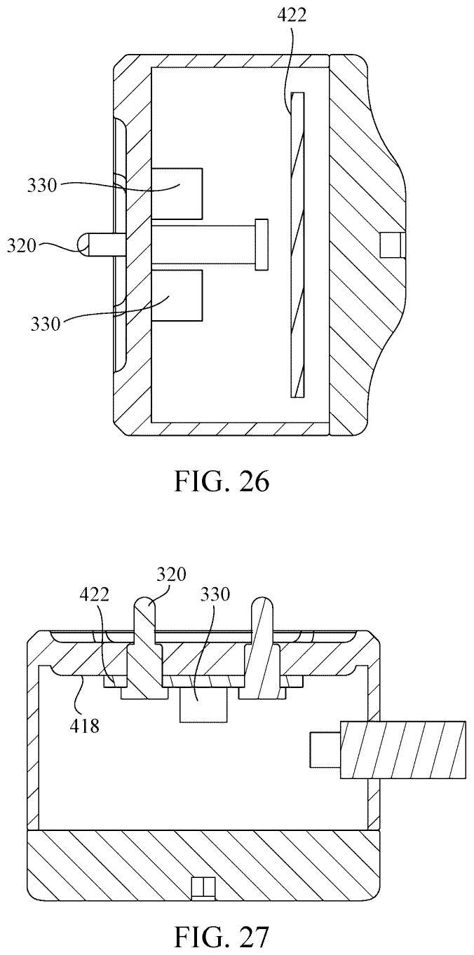

Referring to FIG. 26, the circuit board 422 and the charging terminal 320 are distributed at the axial spacing of the mounting holes 410, so that the circuit board 422 and the charging terminal 320 are electrically connected through a metal sheet or through a wire. The shell 306 is further provided with a refuge hole, a power line 426 is penetrated through the refuge hole. One end of the power line 426 is electrically connected with the circuit board 422, and the other end of the power line 426 is positioned outside the shell 306 and is used for connecting the power supply equipment. The circuit board 422 avoids the refuge hole arrangement in the up-down direction, thereby avoiding interference with the power line 426.

Referring to FIG. 27, in some embodiments, the circuit board 422 is fixed to the inner wall of the recessed portion 404 of the shell 306. Specifically, the inner surface of the shell 306 forms a protruding portion 418 at the position corresponding to the recessed portion 404, and the circuit board 422 is fixed on the protruding portion 418. As the shell 306 is an integral depression to form a recessed portion 404 and a protruding portion 418, the wall thickness of the shell 306 can still be larger at the position corresponding to the recessed portion 404. The wall thickness is not reduced by the setting of the recessed portion 404, so that the circuit board 422 is arranged in the protruding portion 418, and the thicker wall thickness can ensure that the hole is made in the protruding portion 418 to fix the circuit board 422. Moreover, the protruding portion 418 is convex relative to other inner walls, so it can also play a certain guiding and identifying role for the installation of the circuit board 422. Further, the protruding portion 418 may be provided with some positioning posts to be inserted with the positioning holes on the circuit board 422. Alternatively, the protruding portion 418 is provided with a retaining wall and is in contact with the edge of the circuit board 422 to limit the circuit board 422. In this embodiment, the charging terminal 320 may be threaded through the circuit board 422.

The charging connector 302 further comprises a magnetic suction structure 330 such as a magnet. The shell 306 has a first side wall, the recess portion 404 is located at the first side wall, and the magnetic suction structure 330 is also arranged on the first side wall. For instance, it can be located on the outer side or the inner side of the first side wall. The magnetic suction structure 330 is located on the same side wall with the recess portion 404, so that when charging, the magnetic suction structure 330 is at a close distance to the therapy device 428 and has the greatest magnetic attraction, so that the therapy device 428 is fixed, and the therapy device 428 and the charging connector 302 are prevented from being separated.

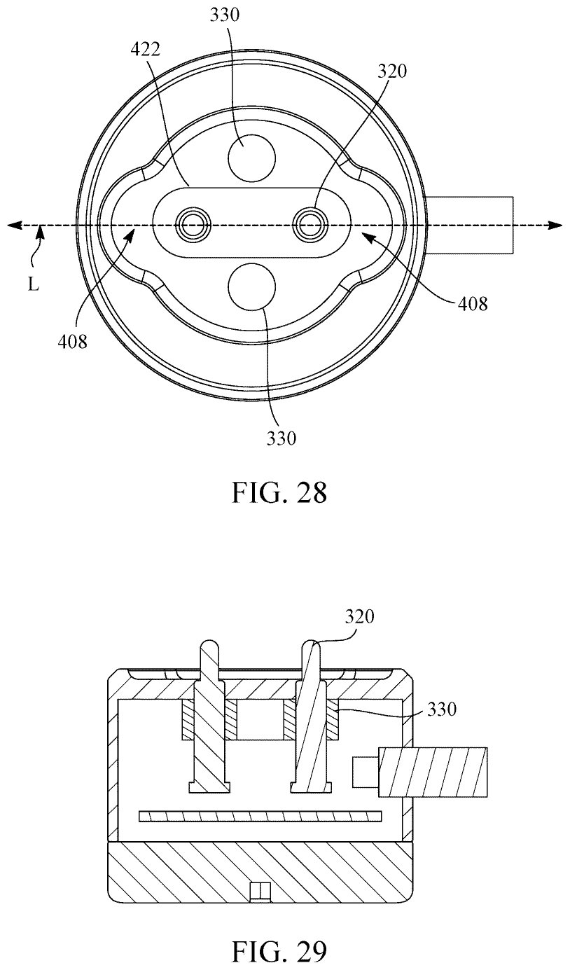

Referring to FIG. 27 and FIG. 28, the charging connector 302 further comprises two magnetic suction structure 330. The two magnetic suction structure 330 are fixed on the inner wall of the recess portion 404 of the shell 306. The distribution direction of the two magnetic suction structure 330 intersects with the distribution direction of the two mounting holes 410 (as shown in FIG. 28). Therefore, the magnetic suction structure 330 avoids the mounting hole 410, and the space of the recessed portion 404 is reasonably utilized, and the compactness of the structure is realized and the interference is avoided. Optionally, the distribution direction of the two magnetic suction structure 330 is perpendicular to the distribution direction of the two mounting holes 410.

In some embodiments, the depression portion 404 has two second grooves 408, and the center lines of the two mounting holes 410 coincide with the center lines of the two second grooves 408. The circuit board 422 is in the shape of a long strip, the length direction of the circuit board 422 (as shown in the direction L shown in FIG. 28) is parallel to the distribution direction of the two second grooves 408. The circuit board 422 is fixed on the inner wall of the corresponding recessed portion 404 of the shell 306. The circuit board 422 is fixed on the inner side of the first side wall, the charging terminal 320 penetrates through the circuit board 422, and the two magnetic suction structure 330 are distributed on two opposite sides of the width direction of the circuit board 422. In this way, the circuit board 422, the magnetic suction structure 330 and the charging terminal 320 are all integrated in the same side wall of the shell 306, and the space is reasonably arranged, and the compact structure is realized. Therefore, the whole charging connector 302 can have a small thickness, has the characteristics of being small, lightweight. When the therapy device 428 is in the vertically upright state, and the charging interface 100 is located at the side of the upper and lower extensions, the miniaturized charging connector 302 can better magnetically attract on the charging interface 100 of the therapy device 428, and avoid its falling.

Correspondingly, the charging interface 100 can also be provided with magnet or metal piece to adsorb with magnetic suction structure 330 on the charging connector 302.

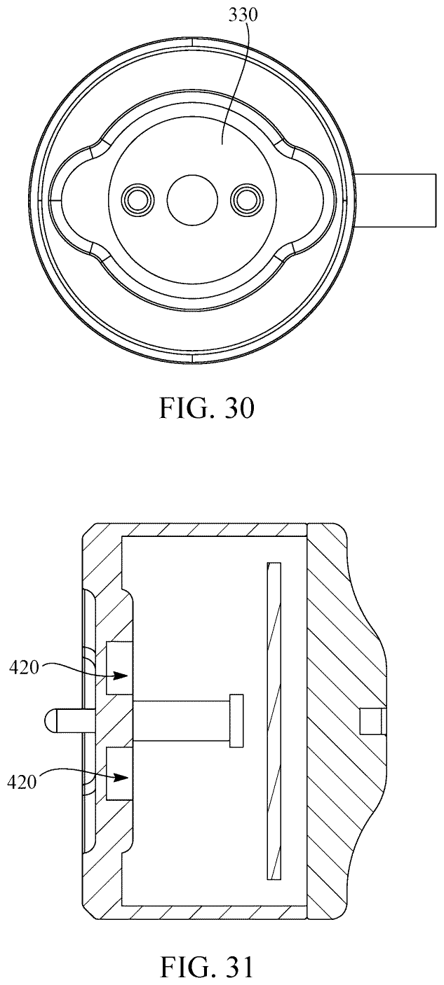

In an embodiment, the number of magnetic suction structure 330 is not limited to two, and can be one or more. When setting one side magnetic suction structure 330, as shown in FIG. 29 and FIG. 30, magnetic suction structure 330 can adopt annular structure.

Referring to FIG. 31, the inner side of the first side wall is provided with a mounting groove 420, and the magnetic suction structure 330 is embedded with the mounting groove 420. In some embodiments, the magnetic suction structure 330 can also be directly bonded to the inside of the first side wall.

The embodiment of the present application also provides a therapy instrument kit. The therapy instrument kit comprises a therapy device 428 and a charging connector 302.

Referring to FIG. 32 and FIG. 33, the therapy device 428 comprises a casing 200, a one or more electro-stimulation element 430, a battery 432, a button and a charging pins 104. The button is fixed with the casing 200. A part of the button is protruding from the outer surface of the casing 200. The charging pins 104 is embedded in the button and is exposed, and the charging pins 104, the charging interface and the one or more electro-stimulation element 430 are electrically connected with the battery 432. The protrusion of the charging interface extends into the recessed portion 404 from the outer surface of the casing 200, and the charging pins 104 is electrically connected with the charging terminal 320. When the button is pressed, can trigger the one or more electro-stimulation element 430 to turn on or off, that is, is equivalent to triggering the one or more electro-stimulation element 430 and the battery 432 to be on or off.

The charging pins 104 and the charging terminal 320 can be structures such as conductive column, conductive sheet, conductive filament, etc.

The foregoing is only an optional embodiment of the present application and is not intended to limit the present application, and any modifications, equivalent substitutions and improvements, etc., made within the spirit and principles of the present application shall be included in the scope of protection of the present application.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A system for providing a therapy to a user, the system comprising:
a therapy device having one or more electro-stimulation element;
a charging interface in the therapy device, the charging interface comprising:
a positioning portion arranged on surface of the therapy device, the positioning portion has one or more positioning hole for positioning a first electrode and a second electrode;
a charging connector having a charging terminal that connects with the first electrode and the second electrode on the charging interface of the therapy device for charging the therapy device; and
wherein the positioning portion act as an activating switch for controlling the one or more electro-stimulation element.

2. The system of claim 1, wherein the electro-stimulation element includes but is not limited to a stimulation element such as Light Emitting Diodes (LEDs), lasers, heating elements, cooling elements, vibration elements, electrodes, micro-current element, an actuator, a pump, a motor, a vibrator, a heater, a piezoelectric element, an abrasive element.

3. The system of claim 1, wherein the charging interface comprises a magnetic suction piece and the charging connector comprises a magnetic suction structure to magnetically attach to the charging interface when docked.

4. A device for providing a therapy to a user, the device comprising:
one or more electro-stimulation element;
a circuit board electrically connected with the one or more electro-stimulation element;
a charging interface, the charging interface comprising:
a positioning portion having one or more positioning hole;
an accommodating cavity;
one or more charging pins in the accommodating cavity for charging the device, the one or more charging pins comprises a first electrode and a second electrode for charging; and
wherein the positioning portion is designed to form a conductive loop between the circuit board and the one or more electro-stimulation element, thereby activating the one or more electro-stimulation element.

5. The device of claim 4, wherein the circuit board is electrically connected with the first electrode and the second electrode, and is used to form a conductive loop when the first electrode and the second electrode are in contact with skin.

6. The device of claim 4, wherein the positioning portion is configured to switch between a first position where the first electrode and the second electrode are in the accommodating cavity, and a second position where the first electrode and the second electrode are exposed to the one or more positioning hole.

7. The device of claim 5, wherein the positioning portion is provided in a casing having a slot and the positioning portion comprise a first positioning hole and a second positioning hole, and the positioning portion comprises:
a positioning wall in the first positioning hole and the second position hole;
a periphery wall configured to move telescopically in the slot;
the periphery wall is connected to an edge of the positioning wall to construct the accommodating cavity, and the accommodating cavity is provided with an opening for the circuit board; and
wherein the positioning wall is configured to move fro and towards the circuit board.

8. The device of claim 7, wherein an elastic part is provided between the positioning wall and the circuit board.

9. The device of claim 7, wherein an inner edge of the opening is provided with a first stop structure on one side of the circuit board, and an outer edge of the periphery wall is provided with a second stop structure.

10. The device of claim 4 further comprising a switch piece connected to the circuit board.

11. The device of claim 10, wherein the positioning portion comprises a boss, wherein when the positioning portion is pressed, the boss presses the switch piece to form a conductive loop between the circuit board and the one or more electro-stimulation element.

12. The device of claim 4, wherein the charging interface further comprises an elastic reset piece connected with a mounting pad, the mounting pad has a movable port for accommodating the positioning portion.

13. The device of claim 12, wherein the positioning portion has a limiting groove and the mounting pad has a limiting portion for inserted in the limiting groove.

14. The device of claim 4, wherein the positioning portion has one or more magnetic suction piece.

15. The device of claim 4, wherein the charging pins are configured to connect to a charging connector for charging the device.

16. The device of claim 4, wherein wall of the charging interface is sealed to make the device waterproof.

17. A charging connector for charging a device for providing therapy to a user, the charging connector comprising:
a shell;
an electrical connection assembly comprises a charging terminal, the charging terminal has a first end in the shell and a second end at a face of the shell;
a magnetic suction structure in the shell or at the charging terminal;
wherein the shell comprises a storage chamber and a recessed portion having two mounting holes, the recessed portion comprises a first groove and a second groove; and
wherein the charging terminal is configured to connect to a charging pin on a charging interface of the device.

18. The charging connector of claim 17, wherein the shell has a first end plate for butting with a charging interface on the device and the magnetic suction structure is positioned at the first end plate.

19. The charging connector of claim 18, wherein the shell has a second end plate opposite to the first end plate, the second end plate has an indicator light connected with the charging terminal.

20. The charging connector of claim 17, wherein the first groove is a circular groove, the second groove is connected to periphery of the first groove, and the mounting holes penetrates through the first groove and communicates with the storage chamber.

21. The charging connector of claim 18, wherein the second groove are two in number, and the center of both the second groove is collinear with the center of the first groove.

22. The charging connector of claim 20, wherein contour of the second groove is arc-shaped.
